(19) United States
(12) Patent Application Publication
(10) Pub. No.: US 2025/0216300 A1
(43) Pub. Date: Jul. 3, 2025

(54) REVERSIBLE FIXING REAGENTS AND METHODS OF USE THEREOF

(71) Applicant: 10x Genomics, Inc., Pleasanton, CA (US)

(72) Inventors: Joshua Delaney, Pleasanton, CA (US); Shalini Gohil, Castro valley, CA (US); Adam Lowe, Bonogin (AU); Yi Luo, Dublin, CA (US); Dagmar Walter, Pleasanton, CA (US)

(21) Appl. No.: 19/022,367

(22) Filed: Jan. 15, 2025

Related U.S. Application Data
(63) Continuation of application No. 17/845,331, filed on Jun. 21, 2022.
Application No. 17/845,331 is a continuation of application No. PCT/US2020/066705, filed on Dec. 22, 2020.
Application No. PCT/US2020/066705 is a continuation of application No. 62/952,677, filed on Dec. 23, 2019.
(60) Provisional application No. 63/026,513, filed on May 18, 2020.

(51) Int. Cl. G01N 1/30; C07D 403/12

(52) U.S. Cl. CPC G01N 1/30; C07D 403/12; G01N 2001/305

(57) ABSTRACT

The present disclosure relates to compositions and methods for reversible fixation of biological samples using fixation reagents that form bis-carbamate crosslinks between amine-bearing moieties in biomolecules.

### CROSS-REFERENCE TO RELATED APPLICATIONS

This application is a continuation of U.S. patent application Ser. No. 17/845,331, filed on Jun. 21, 2022, which claims the benefit of priority to International Application No. PCT/US2020/066705, filed Dec. 22, 2020, U.S. Provisional Application No. 62/952,677, filed Dec. 23, 2019, and to U.S. Provisional Application No. 63/026,513, filed May 18, 2020, each of which are incorporated herein by reference.

### FIELD

The present disclosure relates to fixing reagent compositions comprising bis-imidazole-carboxylate compounds and methods of their use to prepare reversibly fixed biological samples.

### BACKGROUND

Biological samples containing a variety of biomolecules can be processed for various purposes, such as detection of a disease (e.g., cancer) and/or genotyping (e.g., species identification). Microfluidic technologies have been developed to facilitate processing biological samples by partitioning each sample (e.g., cells) into a discrete partition (e.g., a well or a droplet) that is separate from other partitions (e.g., a droplet fluidically isolated from other droplets). This partitioning into discrete partitions enables accurate control of the respective environments interacting with a biological sample. Further, biological samples partitioned in discrete partitions can be barcoded and subjected to chemical or physical processes such as heating, cooling, or chemical reactions. Thus, each discrete partition can contain its own separate sample and subject it to its own separate assay that can be qualitatively or quantitatively processed.

Biological samples, however, are unstable. When a biological sample is removed from its viable niche physical decomposition begins immediately. The degree of decomposition is determined by a number of factors including time, solution buffering conditions, temperature, source (e.g., certain tissues and cells a have higher levels of endogenous RNase activity), biological stress (e.g., enzymatic tissue dissociation can activate stress response genes), and physical manipulation (e.g. pipetting, centrifuging). The degradation includes important nucleic acid molecules (e.g., RNA), proteins, as well as higher-order 3D structure of molecular complexes, whole cells, tissues, organs, and organisms. The instability of biological samples is a significant obstacle for their use with partition-based assays (e.g., droplet-based or well-based single cell assays). Sample degradation greatly limits the ability to use such assays accurately and reproducibly with a wide range of available biological samples.

The problem of biological sample instability can be mitigated by fixing the sample using standard methods such as cryopreservation, dehydration (e.g., in methanol), high-salt storage (e.g., using RNAssist or RNAlater), and/or chemical fixing agents that create covalent crosslinks (e.g., paraformaldehyde or DSP). The ability to use such a fixed biological sample in an assay, particularly a partition-based assay, requires that the fixed biological sample can be rapidly and efficiently un-fixed, so that the relevant assay can be carried out before sample degradation occurs.

There remains a need for compositions and methods that provide reversible fixation of biological samples in a manner compatible with partition-based assays (e.g., microfluidic droplet-based assays or well-based assays).

### SUMMARY

The present disclosure provides compositions and methods that allow for the reversible fixation of biological samples, and the use of such compositions and methods in partition-based assays (e.g., well- or droplet-based assays), such as gene expression profiling assays.

In at least one embodiment, the present disclosure provides a method for preparing a biological sample comprising contacting the sample with a first fixing reagent composition comprising a compound of formula (I)

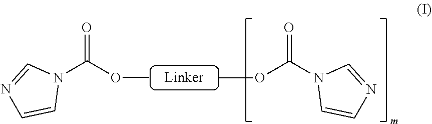

wherein, the “Linker” comprises an ethylene glycol moiety, and/or a linear or branched alkane moiety of 2-24 carbons; and m is 1 to 12; optionally, wherein m=1. In at least one embodiment, the “Linker” comprises: (a) a linear alkane moiety of 2-24 carbons; (b) an ethylene glycol moiety; (c) a disulfide bond; and/or (d) a branched alkane moiety.

In at least one embodiment of the method the compound of formula (I) is a compound of formula (II)

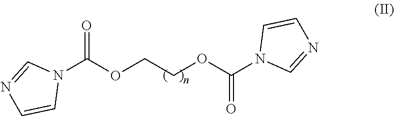

wherein n is 1 to 13.

In at least one embodiment of the method the compound of formula (I) is a compound of formula (III)

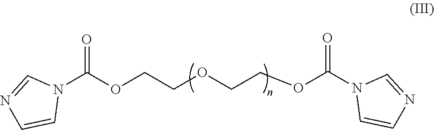

wherein n is 1 to 12.

In at least one embodiment of the method the compound of formula (I) is a compound of formula (IV)

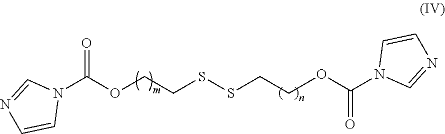

wherein m is 1 to 13, and n is 1 to 13.

In at least one embodiment of the method the compound of formula (I) is a compound of formula (V)

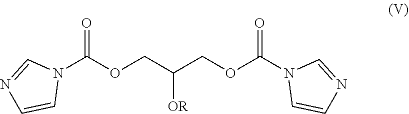

wherein R is selected from —H, —O(CO)—CH₃, and —O(CO)-imidazole.

In at least one embodiment of the method the compound of formula (I) is compound (6)

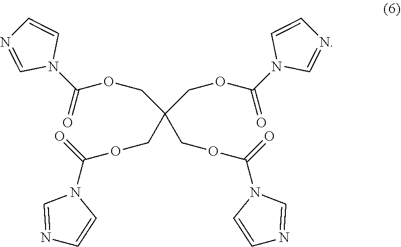 [SMILES: O=C(OCC(COC(=O)n1ccnc1)(COC(=O)n1ccnc1)COC(=O)n1ccnc1)n1ccnc1]

In at least one embodiment of the method, the first fixing reagent composition comprises: (a) the compound of formula (I) at a concentration of 50 mM or less, 25 mM or less, 15 mM or less, 10 mM or less, or 5 mM or less; and/or (b) DMSO at a concentration of 5% or less, 2.5% or less, or 1.5% or less.

In at least one embodiment of the method, contacting with the first fixing reagent composition at room temperature (RT) is for 3 h or less, 60 min or less, 30 min or less, 15 min or less, or 5 min or less.

In at least one embodiment the method further comprises contacting the sample with a second fixing reagent composition; optionally, wherein the second fixing reagent composition comprises paraformaldehyde; optionally, wherein the paraformaldehyde concentration is 1% or less.

In at least one embodiment of the method, the first fixing reagent composition comprises a compound of formula (II)

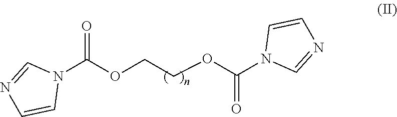

wherein n is 1 to 13; and the second fixing reagent composition comprises a compound of formula (III):

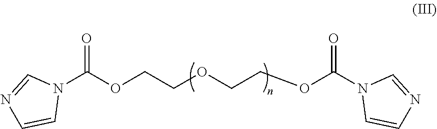

wherein n is 1 to 12.

In at least one embodiment of the method, the first fixing reagent composition comprises a compound of formula (III), wherein n is 1 to 12; and the second fixing reagent composition comprises a compound of formula (II), wherein n is 1 to 13.

In at least one embodiment of the method, the method further comprises contacting the sample with an unfixing agent; optionally, wherein the unfixing agent comprises: (a) a compound capable of cleaving a carbamate bond; optionally, wherein the compound capable of cleaving a carbamate bond selected from DETA, EDA, hydrazine monohydrate, a carboxyesterase, or a combination thereof; (b) a compound capable of cleaving a disulfide bond; optionally, wherein the compound capable of cleaving a disulfide bond is DTT; and/or (c) a compound capable of reversing paraformaldehyde fixation.

In at least one embodiment, the present disclosure also provides a composition comprising a fixed biological sample, wherein the sample comprises crosslinked biomolecules of formula (Ia)

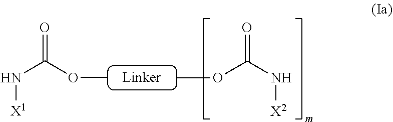

wherein, X¹ and X² are amine-bearing moieties of the same or different biomolecules of the sample; and “Linker” comprises an ethylene glycol moiety, and/or a linear or branched alkane moiety of 2-24 carbons; and m is 1 to 12. In at least one embodiment, the “Linker” comprises: (a) a linear alkane moiety of 2-24 carbons; (b) an ethylene glycol moiety; (c) a disulfide bond; and/or (d) a branched alkane moiety.

In at least one embodiment of the composition, the fixed biological sample comprises crosslinked biomolecules of formula (IIa)

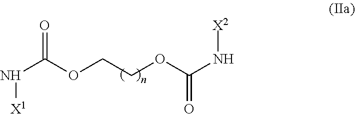

wherein, n is 1 to 13.

In at least one embodiment of the composition, the fixed biological sample comprises crosslinked biomolecules of formula (IIIa)

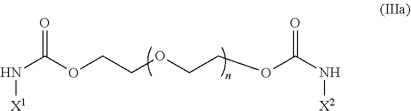

wherein, n is 1 to 12.

In at least one embodiment of the composition, the fixed biological sample comprises crosslinked biomolecules of formula (IVa)

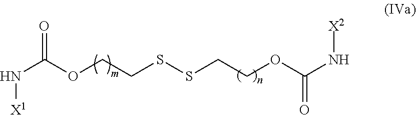

wherein m is 1 to 13, and n is 1 to 13.

In at least one embodiment of the composition, the fixed biological sample comprises crosslinked biomolecules of formula (Va)

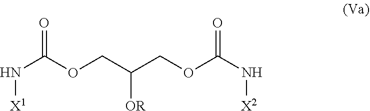

wherein R is selected from —H, —O(CO)—CH₃, and —O(CO)-imidazole.

In at least one embodiment of the composition, the fixed biological sample comprises crosslinked biomolecules of formula (VIa)

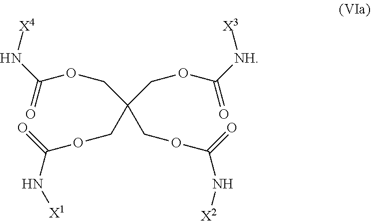

In at least one embodiment of the composition, the fixed biological sample is derived from a tissue sample, a biopsy sample, or a blood sample; optionally, wherein the fixed biological sample is a single cell.

In at least one embodiment of the composition, the fixed biological sample has been fixed with a fixing reagent composition comprising a compound of any one of formulas (I), (II), (III), (IV), (V), or compounds (6).

In at least one embodiment of the composition, the fixed biological sample is provided in a discrete partition (e.g., provided or encapsulated in a discrete droplet or provided in a well) with a lysis and/or an unfixing agent; optionally, wherein the unfixing agent comprises a compound capable of cleaving a carbamate bond; optionally, wherein the compound capable of cleaving a carbamate bond selected from DETA, EDA, hydrazine monohydrate, a carboxyesterase, or a combination thereof.

In at least one embodiment of the composition, the fixed biological sample is provided in a discrete partition (e.g., provided or encapsulated in a discrete droplet or provided in a well), wherein the discrete partition further comprises a support (e.g., a bead); optionally, wherein the unfixing agent is contained in a support (e.g., a bead).

In at least one embodiment of the composition, the fixed biological sample is provided in a discrete partition (e.g., provided or encapsulated in a discrete droplet or provided in a discrete well), wherein the discrete partition further comprises assay reagents; optionally, wherein the assay reagents are provided as part of a support (e.g., a bead) or contained in a support (e.g., a bead).

In at least one embodiment of the composition, the fixed biological sample is provided in a discrete partition (e.g., provided or encapsulated in a discrete droplet or provided in a discrete well), wherein the discrete partition further comprises a barcode optionally, wherein the barcode is contained as part of a support (e.g., a bead).

In at least one embodiment, the present disclosure also provides an assay method comprising: (a) generating a discrete partition comprising a fixed biological sample (e.g., a droplet comprising or encapsulating a fixed biological sample), a lysis and/or an unfixing agent, and assay reagents, wherein the fixed sample comprises crosslinks of formula (Ia)

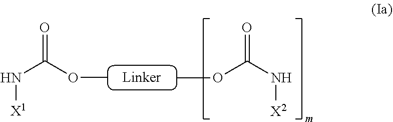

wherein, X¹ and X² are amine-bearing moieties of the same or different biomolecules of the sample; the “Linker” comprises an ethylene glycol moiety, and/or a linear or branched alkane moiety of 2-24 carbons; and m is 1 to 12; the unfixing agent comprises a compound capable of cleaving a carbamate bond; optionally, wherein the compound capable of cleaving a carbamate bond selected from DETA, EDA, hydrazine monohydrate, a carboxyesterase, or a combination thereof; and (b) detecting analytes from the reaction of the assay reagents and the un-fixed biological sample.

In at least one embodiment, the present disclosure also provides a kit comprising: assay reagents; and a fixing reagent composition comprising a compound of formula (I)

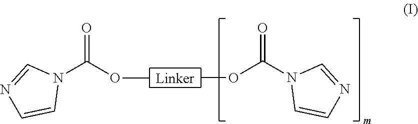

wherein, the “Linker” comprises an ethylene glycol moiety, and/or a linear or branched alkane moiety of 2-24 carbons; and m is 1 to 12. In at least one embodiment, the “Linker” comprises: (a) a linear alkane moiety of 2-24 carbons; (b) an ethylene glycol moiety; (c) a disulfide bond; and/or (d) a branched alkane moiety. In at least one embodiment, the fixing reagent composition comprises a compound of any one of formulas (II), (III), (IV), (V), or compound (6).

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7 depicts plots of cell counting of different PBMC cell types found in fresh cells as compared to cells fixed with compound (2a), stored for 3 days, and then subjected to the un-fixing treatment with CES and hydrazine, as described in Example 7.

FIG. 8 shows an exemplary barcode carrying bead.

FIG. 9 shows another exemplary barcode carrying bead.

FIG. 10 shows an exemplary microwell array schematic.

FIG. 11 shows an exemplary microwell array workflow for processing nucleic acid molecules.

FIG. 12 schematically illustrates examples of labelling agents.

FIG. 13 depicts an example of a barcode carrying bead.

FIGS. 14A, 14B, and 14C schematically depict an example workflow for processing nucleic acid molecules.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the embodiments of the disclosure. The upper and lower limits of these smaller ranges may independently be included in the smaller ranges, and are also encompassed within the embodiments of the disclosure, subject to any specifically excluded limit in the stated range. Where the stated range includes one or both of these limits, ranges excluding (i) either or (ii) both of those included limits are also included in the embodiments of the disclosure. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present disclosure pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### A. Fixing Reagent Compositions of Bis-Imidazole-Carboxylate Compounds

The present disclosure provides compositions and methods that allow for the reversible fixation of biological samples. The ability to reversible fix biological samples is based on the use of a fixing reagent composition comprising a bis-imidazole-carboxylate compound of general formula (I):

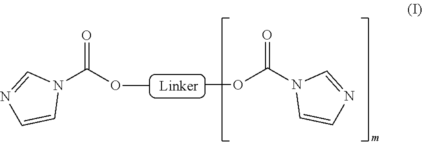

The “Linker” depicted schematically in formula (I) can be any chemical group capable of covalently linking the imidazole carboxylate moieties. In at least one embodiment of the compound of formula (I), “Linker” comprises an ethylene glycol moiety, and/or a linear or branched alkane moiety of 2-24 carbons; and m is 1 to 12.

The compounds of formula (I) can be prepared by reacting carbonyl diimidazole (CDI) with a desired bis-hydroxy linker compound as depicted in Scheme 1.

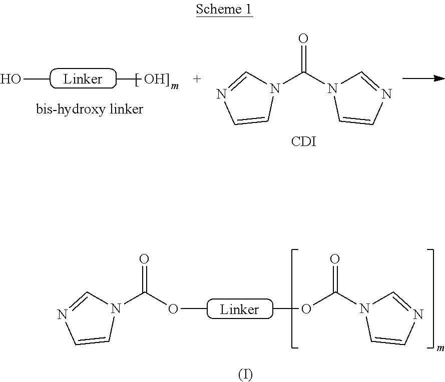

Typical CDI synthesis reaction conditions can be used, e.g., 1.5-fold to 4-fold excess CDI in dichloromethane, 16 h at RT, as described in Example 1. The general CDI-based reaction of Scheme 1 can be used with a wide range of bis-hydroxy linker compounds (e.g., glycerol, bis-hydroxy-alkanes, PEG compounds, hydroxy-substituted branched-alkyl compounds) to provide a wide range of fixation reagent compounds of formula (I).

Accordingly, in at least one embodiment, the present disclosure also provides a fixing reagent composition, wherein the compound of formula (I) is selected from:
(a) a compound of formula (II)

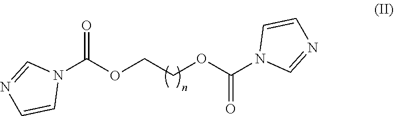

wherein n is 1 to 13;
(b) a compound of formula (III)

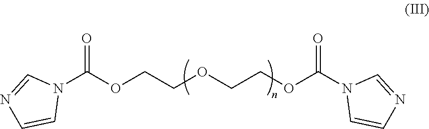

wherein, n is 1 to 12;
(c) a compound of formula (IV)

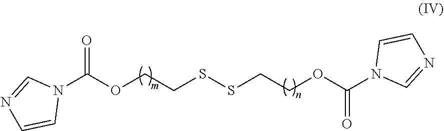

wherein m is 1 to 13, and n is 1 to 13;
(d) a compound of formula (V)

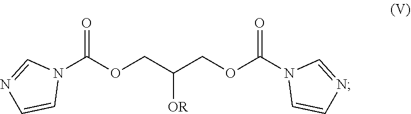

wherein R is —H, —O—(CO)—CH₃, or —O—(CO)-imidazole
   or
(e) a compound (6)

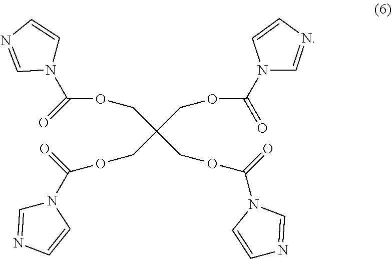 [SMILES: O=C(OCC(COC(=O)n1ccnc1)(COC(=O)n1ccnc1)COC(=O)n1ccnc1)n1ccnc1]

Additional specific bis-imidazole-carboxylate compounds of formulas (I), (II), (III), (IV), and (V) are contemplated. A non-exhaustive list of such compounds useful as fixing reagents in the compositions and methods of the present disclosure are provided in Table 1.

TABLE 1

| | |
|---|---|
| 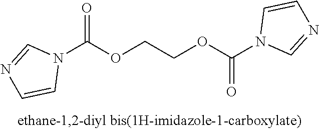 [SMILES: O=C(OCCOC(=O)n1ccnc1)n1ccnc1] | (2a) |
| 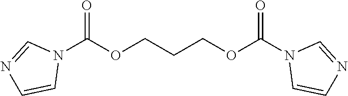 [SMILES: O=C(OCCCOC(=O)n1ccnc1)n1ccnc1] | (2b) |
| 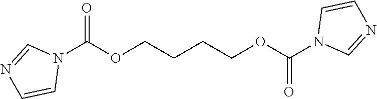 [SMILES: O=C(OCCCCOC(=O)n1ccnc1)n1ccnc1] | (2c) |
| 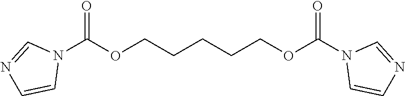 [SMILES: O=C(OCCCCCOC(=O)n1ccnc1)n1ccnc1] | (2d) |
| 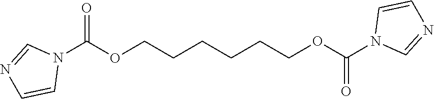 [SMILES: O=C(OCCCCCCOC(=O)n1ccnc1)n1ccnc1] | (2e) |
| 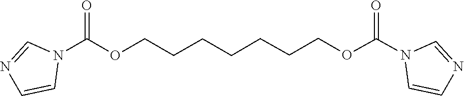 [SMILES: O=C(OCCCCCCCOC(=O)n1ccnc1)n1ccnc1] | (2f) |
| 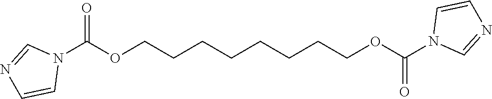 [SMILES: O=C(OCCCCCCCCOC(=O)n1ccnc1)n1ccnc1] | (2g) |
| 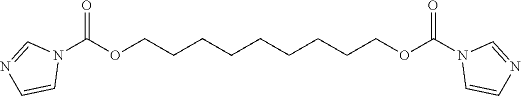 [SMILES: O=C(OCCCCCCCCCOC(=O)n1ccnc1)n1ccnc1] | (2h) |
| 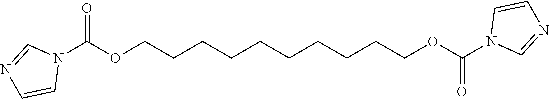 [SMILES: O=C(OCCCCCCCCCCOC(=O)n1ccnc1)n1ccnc1] | (2i) |
| 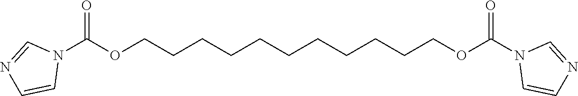 [SMILES: O=C(OCCCCCCCCCCCOC(=O)n1ccnc1)n1ccnc1] | (2j) |
| 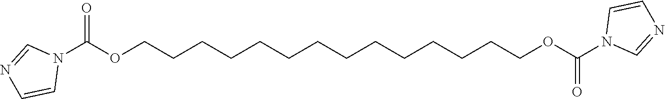 [SMILES: O=C(OCCCCCCCCCCCCCCOC(=O)n1ccnc1)n1ccnc1] | (2k) |
| 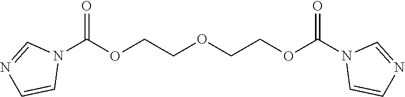 [SMILES: O=C(OCCOCCOC(=O)n1ccnc1)n1ccnc1] | (3a) |
| 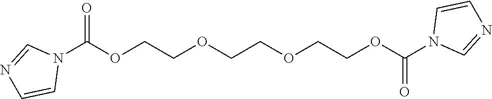 [SMILES: O=C(OCCOCCOCCOC(=O)n1ccnc1)n1ccnc1] | (3b) |
| 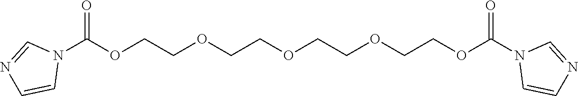 [SMILES: O=C(OCCOCCOCCOCCOC(=O)n1ccnc1)n1ccnc1] | (3c) |
| 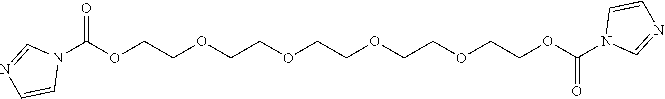 [SMILES: O=C(OCCOCCOCCOCCOCCOC(=O)n1ccnc1)n1ccnc1] | (3d) |
| 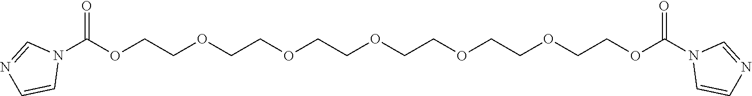 [SMILES: O=C(OCCOCCOCCOCCOCCOCCOC(=O)n1ccnc1)n1ccnc1] | (3e) |
| 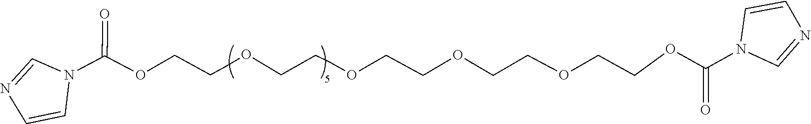 | (3f) |
| 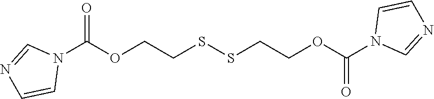 [SMILES: O=C(OCCSSCCOC(=O)n1ccnc1)n1ccnc1] | (4a) |
| 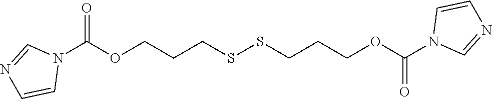 [SMILES: O=C(OCCCSSCCCOC(=O)n1ccnc1)n1ccnc1] | (4b) |
| 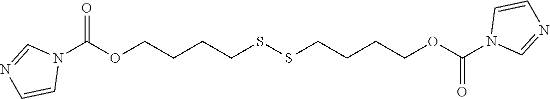 [SMILES: O=C(OCCCCSSCCCCOC(=O)n1ccnc1)n1ccnc1] | (4c) |
| 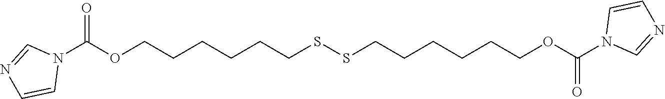 [SMILES: O=C(OCCCCCCSSCCCCCCOC(=O)n1ccnc1)n1ccnc1] | (4d) |
| 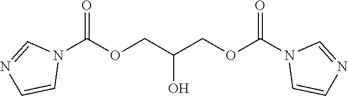 [SMILES: O=C(OCC(O)COC(=O)n1ccnc1)n1ccnc1] | (5a) |
| 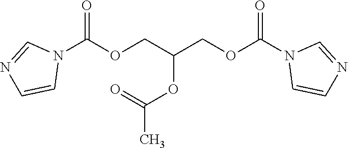 [SMILES: CC(=O)OC(COC(=O)n1ccnc1)COC(=O)n1ccnc1] | (5b) |
| 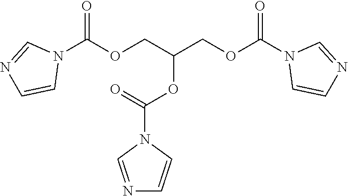 [SMILES: O=C(OCC(COC(=O)n1ccnc1)OC(=O)n1ccnc1)n1ccnc1] | (5c) |

### B. Use of Bis-Imidazole-Carboxylate Compounds to Prepare Biological Samples

The bis-imidazole-carboxylate compounds of formula (I) (and sub-genus compounds of formulas (II), (III), (IV), (V) and Table 1) provide the advantageous technical effect of rapidly and efficiently reacting with the amine-bearing moieties of biomolecules in a biological sample (e.g., protein lysine and arginine side-chains; nucleic acid adenine and guanine nucleobases) to form bis-carbamate crosslinks. A generalized biological sample fixation reaction using a fixation reagent of formula (I) is shown in Scheme 2.

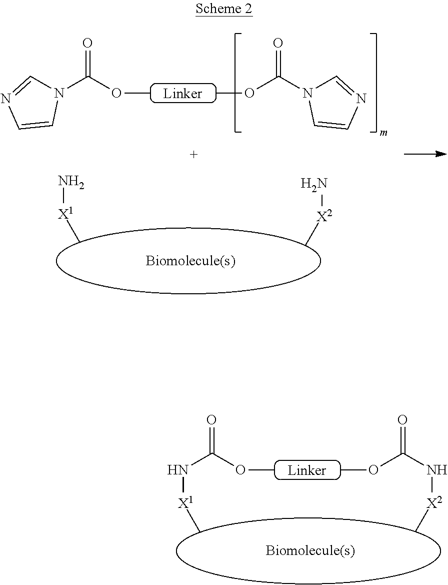

As used herein, the term “biological sample,” refers to any sample of biological origin that includes a biomolecule. The term “biomolecule” is intended to encompass proteins, peptides, nucleic acids, carbohydrates, lipids, and all other biological macromolecules. Biological samples used in the methods and compositions of the present disclosure include blood and other liquid samples of biological origin, solid tissue samples such as a tissue sample (i.e., tissue specimen), a biopsy (i.e., a biopsy specimen), or tissue cultures or cells derived therefrom and the progeny thereof. This includes samples that have been manipulated in any way after isolation from the biological source, such as by treatment with reagents (e.g., fixation reagents, thereby generating a fixed biological sample); samples such as tissues that are embedded in medium (e.g., paraffin); sectioned tissue sample (e.g., sectioned samples that are mounted on a solid substrate such as a glass slide); washed; or enrichment for certain cell populations, such as cancer cells, neurons, stem cells, etc. The term also encompasses samples that have been enriched for particular types of molecules, e.g., nucleic acids, polypeptides, etc. Biological sample is also intended to include a clinical sample, including tissue obtained by surgical resection, tissue obtained by biopsy, cells in culture, cell supernatants, cell lysates, tissue samples (i.e., tissue specimens), organs, bone marrow, blood, plasma, serum, and the like. A biological sample can also include a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample having cells (e.g., cancer cells) from a patient.

The term “fixed” as used herein with regard to biological samples refers to a state of being preserved from decay and/or degradation. “Fixation” refers to a process that results in a fixed sample, and can include contacting the biomolecules within a biological sample with a fixation reagent (or “fixative”) for some amount of time, whereby the fixation reagent reacts with the biomolecules of the sample to form covalent crosslinks between chemical moieties of the biomolecules (e.g., amine-bearing moieties). The reaction of the bis-imidazole-carboxylate fixation reagents with the amine-bearing moieties of biomolecules is described in greater detail below.

Herein, “un-fixed” refers to the processed condition of a cell, a plurality of cells, a tissue sample or any other biological sample that is characterized by a prior state of fixation followed by a reversal of the prior state of fixation. For instance, an un-fixed cell may also be referred to as a “previously fixed” cell. In one embodiment, an un-fixed cell is characterized by broken or reversed covalent bonds in the biomolecules of the cell(s) or sample, where such covalent bonds were previously formed by treatment with a fixation agent described herein.

The general biological sample fixation reaction of Scheme 2 can be carried out using a fixing reagent composition comprising compounds of formula (I) under standard aqueous conditions typically used for fixation of biological samples. The amount of time a biological sample is contacted with a fixative to provide a fixed biological sample depend on the temperature, the nature of the sample, and the fixative used. For example, as described elsewhere herein and the Examples an isolated sample of cells is incubated with an aliquot of a solution of the compound (2a) at a concentration of e.g., 5-15 mM for 30-60 minutes at room temperature, before quenching with excess buffer. The resulting fixed biological sample is preserved and can be stored under typical cryopreservation conditions for days, weeks, or months, before use.

Accordingly, in at least embodiment, the present disclosure provides a method for preparing a biological sample comprising contacting the sample with a first fixing reagent composition comprising a compound of formula (I)

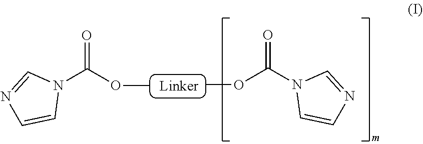

wherein, “Linker” comprises an ethylene glycol moiety, and/or a linear or branched alkane moiety of 2-24 carbons; and m is 1 to 12. It is further contemplated that the methods can be carried out wherein the compound of formula (I) is a compound of any of formulas (II), (III), (IV), or (V), or compound (6), or any one or more of the bis-imidazole-carboxylate compounds disclosed in Table 1.

For example, a biological fixation reaction of the method can be carried out using a fixing reagent compound of formula (IV) as depicted Scheme 3.

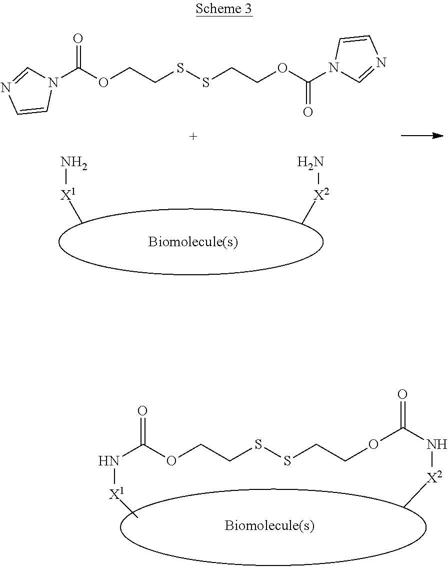

Similarly, a biological fixation reaction can be carried out using a fixation reagent compound of formula (I) wherein m=3. Such a reaction, which can be carried out using a reagent such as compound (6), results in branched bis-carbamate crosslinks between biomolecules of the biological samples, as shown by the generalized reaction of Scheme 4,

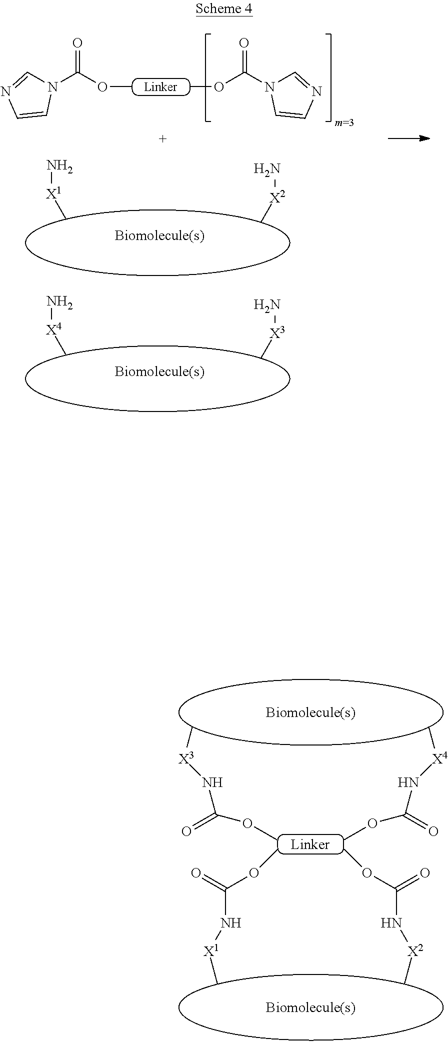

### C. Methods Using Combinations of Fixing Reagents

It is also contemplated that a mixture of the bis-imidazole carboxylate fixing reagent compounds of formula (I) can be used in the methods of the present disclosure. For example, in at least one embodiment, the fixing reagent composition comprises a compound of formula (II), which comprises a Linker having an alkyl chain, and a compound of formula (III), which comprising a Linker having an ethylene glycol chain. Without being bound by theory, it is contemplated that the compounds of formula (III) having an ethylene glycol chain “Linker” moiety are more soluble in aqueous solution and well-suited for fixing biomolecules on the surfaces of cells. In contrast, it is contemplated that the fixing reagent compounds of formula (II), which comprise a more hydrophobic alkyl chain in the Linker moiety are less soluble in aqueous environments and better suited for fixing biomolecules in interior of a cell. Accordingly, in at least one embodiment, the method can be carried out wherein the method comprises contacting the sample with a first fixing reagent composition comprising a compound of formula (II) and a compound of formula (III). Generally, the selection of the particular bis-imidazole-carboxylate compounds to be used in a fixing reagent composition useful in methods for preparing a biological sample is guided by factors typically considered in the development of sample preparation methods. Such factors include, but are not limited to, reagent solubility and compatibility with the type of biological sample, the conditions used, and the desired amount of fixation/preservation.

Further, the bis-imidazole carboxylate fixing reagent compounds of formula (I) can be used in combination with a second fixing reagent compound that does not form bis-carboxylate crosslinks. Examples of fixing reagent compounds that can be used in combination with the bis-imidazole carboxylate fixing reagent compounds of formula (I) include but are not limited to aldehyde fixatives (e.g., formaldehyde, also commonly referred to as “paraformaldehyde” and “formalin”; glutaraldehyde; etc.), imidoesters, NHS (N-Hydroxysuccinimide) esters, and the like.

In some embodiments, the second fixing reagent useful in the methods of the present disclosure is paraformaldehyde (or “PFA”). Generally, the term “paraformaldehyde” in the context of a fixative is used interchangeably with “formaldehyde” and “formalin.” Thus, a PFA-fixed biological sample may also be referred to as formalin-fixed or formaldehyde-fixed. Protocols and methods for the use of PFA as a fixation reagent with biological samples are well known in the art, and can be used in the methods and compositions of the present disclosure.

PFA is a strong fixative that crosslinks the nucleobases of nucleic acids in a sample with aminal bonds that are difficult to reverse (or un-fix) under conditions that also maintain the integrity of the nucleic acids for further analysis—e.g., in an RNA profiling assay. Typically, a fixing reagent solution of 4% PFA is used for preparing biological samples. It is contemplated that a significantly lower concentration of PFA can be used in a method of preparing a biological sample when used in combination with a bis-imidazole carboxylate fixing reagent compounds of formula (I). For example, a biological sample can be treated with first fixing reagent composition comprising compound (2a), then treated with a second fixing reagent composition comprising a 1% PFA. Without intending to be bound by theory, it is believed that the initial treatment with the bis-imidazole carboxylate fixing reagent of compound (2a) results in the fixation of available mRNA with DETA-reversible bis-carbamate crosslinks, and the secondary treatment with 1% PFA stabilizes proteins and other biomolecules with aminal crosslinks. The use of only 1% PFA, however, provides sufficient sample stability in view of the prior treatment with compound (2a), for some period of time (e.g., >7 days), and also allows for sufficient ability to un-fix the sample and carry out assays, such as RNA expression profiling. Accordingly, in at least one embodiment, the present disclosure provides a method for preparing a biological sample comprising contacting the sample with a first fixing reagent composition comprising a compound of formula (I) and a second fixing reagent composition, wherein the second fixing reagent composition comprises PFA at concentration of 2% or less, 1% or less, or 0.5% or less.

Conditions for reversing the effects of PFA fixation of a biological sample are known in the art, however, these conditions tend to be harsh. See e.g., WO2001/46402; US2005/0014203A1, and US2009/0202998A1. For example, treatment of PFA-treated tissue samples includes heating to 60-70 C in Tris buffer for several hours, and yet typically results in removal of only a fraction of the PFA-induced crosslinks. The use of such harsh un-fixing treatment conditions can result in permanent damage to biomolecules, particularly nucleic acids, in the sample. Less harsh techniques and conditions have been proposed that utilize un-fixing compounds capable of catalytically cleaving the aminal bond that result from PFA fixation are described in e.g., Karmakar et al., “Organocatalytic removal of formaldehyde adducts from RNA and DNA bases,” Nature Chemistry, 7:752-758 (2015); US 2017/0283860A1; and US 2019/0135774A1. . . . See e.g., Karmakar et al., “Organocatalytic removal of formaldehyde adducts from RNA and DNA bases,” Nature Chemistry, 7:752-758 (2015); US 2017/0283860A1; and US 2019/0135774A1. Accordingly, in at least one embodiment of the methods of the present disclosure it is contemplated that the method can further comprise use of an catalytic unfixing agent compound capable reversing PFA fixation. Exemplary catalytic compounds capable of reversing PFA fixation are disclosed in Table 2 below.

TABLE 2

| | |
|---|---|
| 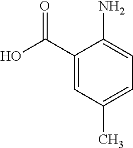 | (7a) |
| 2-amino-5-methylbenzoic acid | |
| (CAS No. 2941-78-8; Sigma-Aldrich) | |
| 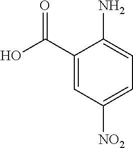 [SMILES: Nc1ccc([N+](=O)[O-])cc1C(=O)O] | (7b) |
| 2-amino-5-nitrobenzoic acid | |
| (CAS No. 616-79-5; Sigma-Aldrich) | |
| 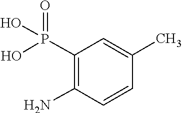 | (7c) |
| (2-amino-5-methylphenyl)phosphonic acid | |
| (CAS 69675-98-5; Ambeed Inc.) | |
| 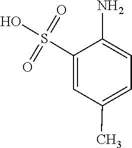 | (7d) |
| 2-amino-5-methylbenzenesulfonic acid | |
| (CAS No. 88-44-8; Sigma-Aldrich) | |
| 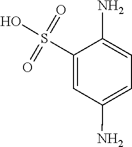 [SMILES: Nc1ccc(N)c(S(=O)(=O)O)c1] | (7e) |
| 2,5-diaminobenzenesulfonic acid | |
| (CAS No. 88-45-9; Sigma-Aldrich) | |
| 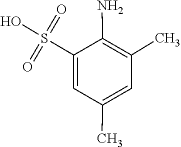 | (7f) |
| 2-amino-3,5-dimethylbenzenesulfonic acid | |
| (CAS No. 88-22-2; TCI Co. Ltd., Tokyo, JP) | |
| 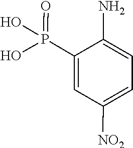 [SMILES: Nc1ccc([N+](=O)[O-])cc1P(=O)(O)O] | (7g) |
| (2-amino-5-nitrophenyl)phosphonic acid | |
| 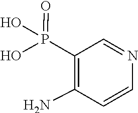 [SMILES: Nc1ccncc1P(=O)(O)O] | (7h) |
| (4-aminopyridin-3-yl)phosphonic acid | |
| 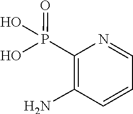 [SMILES: Nc1cccnc1P(=O)(O)O] | (7i) |
| (3-aminopyridin-2-yl)phosphonic acid | |
| 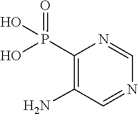 [SMILES: Nc1cncnc1P(=O)(O)O] | (7j) |
| (5-aminopyrimidin-4-yl)phosphonic acid | |
| 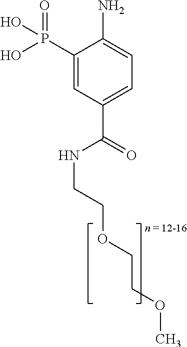 | (7k) |
| (2-amino-5-{[2-(2-poly-ethoxy)ethyl]carbamoyl} | |
| phenyl)phosphonic acid | |
| 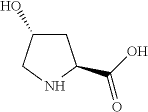 | (7l) |
| (2S,4R)-4-hydroxypyrrolidine-2-carboxylic acid | |
| (″trans-4-hydroxy-L-proline;″ CAS No. 51-35-4; Sigma-Aldrich) | |
| 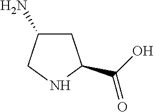 | (7m) |
| (2S,4R)-4-aminopyrrolidine-2-carboxylic acid | |
| (″trans-4-aminoproline;″ CAS No. 16257-88-8) | |
| 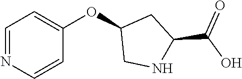 [SMILES: O=C(O)[C@@H]1C[C@H](Oc2ccncc2)CN1] | (7n) |
| (2S,4S)-4-[(pyridin-4-yl)oxy]pyrrolidine-2-carboxylic acid | |
| (CAS No. 2309431-82-9; Enamine Ltd.) | |
| 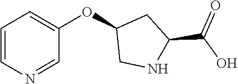 [SMILES: O=C(O)[C@@H]1C[C@H](Oc2cccnc2)CN1] | (7o) |
| (2S,4S)-4-[(pyridin-3-yl)oxy]pyrrolidine-2-carboxylic acid | |
| (″cis-m-O-Py-Pro″) | |

Compounds (7a)-(7f), (7l), and (7n) are commercially available. The compounds (7g), (7h), (7i), (7j), (7k), (7m), and (70) can be prepared from commercially available reagents using standard chemical synthesis techniques well-known in the art. See e.g., Crisalli et al., “Importance of ortho Proton Donors in Catalysis of Hydrazone Formation,” Org. Lett. 2013, 15, 7, 1646-1649.

Briefly, compound (7h) can be prepared in a 2-step synthesis from a commercially available compound as follows. Step 1: Diethyl (4-aminopyridin-3-yl) phosphonate is prepared according to the procedure described in Guilard, R. et al. Synthesis, 2008, 10, 1575-1579. Briefly, to a solution of 3-bromopyridine-4-amine (2.5 g, 14.5 mmol, 1 equiv) (CAS: 13534-98-0, Sigma Aldrich) in ethanol (58 mL) is added diethyl phosphite (2.2 mL, 17.3 mmol, 1.2 equiv.) triethylamine (3 mL, 1.5 equiv), PPh3 (1.1 g, 4.3 mmol, 30 mol %) and Pd(OAc)₂ (0.39 g, 1.73mmol, 12 mol %). The reaction mixture is purged with Argon for 5 min. After heating to reflux for 24 h, the reaction mixture is cooled to room T and concentrated in vacuo. The residue is purified by silica gel chromatography (MeOH/DCM) to yield diethyl (4-aminopyridin-3-yl)phosphonate (¹H NMR (80 MHz, CDCl₃): δ=1.15 (t, 6H, CH₃), 4.18-3.69 (m, 4H, CH₂), 5.99 (br-s, 2H, NH₂), 6.49 (d, 1H), 8.03-7.93 (m, 1H), 8.22 (d, 1H)). Step 2: The precursor compound of Step 1, diethyl (4-aminopyridin-3-yl) phosphonate (0.35 g, 1.52 mmol, 1 equiv) is suspended in 6 N HCl (aq.) (8 mL). After refluxing for 12 h, the reaction mixture is concentrated in vacuo. The residue is washed with DCM, ether and conc in vacuo to afford the target 4-aminopyridin-3-yl-phosphonic acid of compound (7h) (¹H NMR (80 MHz, D₂O): δ=6.85-6.55 (m, 1H), 8.05-7.94 (m, 1H), 8.40-8.26 (m, 1H)0.

Compound (7k) can be prepared in a 4-step synthesis from a commercially available compound as follows. Step 1: To a solution of methyl 4-amino-3-iodobenzoate (2 g, 7.2 mmol, 1 equiv) (CAS: 19718-49-1, Sigma Aldrich) in acetonitrile (20 mL) is added triethyl phosphite (1.9 mL, 10.8 mmol, 1.5 equiv.) and Pd(OAc)₂ (0.16 g, 0.72 mmol, 10 mol %). This reaction mixture is purged with Argon for 5 min. After heating to reflux for 18 h, the reaction mixture is cooled to room temperature and conc. in vacuo. The residue was partitioned between ethyl acetate and water, and the organic layer was dried with MgSO₄ and conc. in vacuo. The crude mixture was purified by silica gel chromatography (ethyl acetate/hexane) to yield methyl-4-amino-3-(diethoxyphosphoryl)benzoate (¹H NMR (500 MHz, DMSO-d₆): δ=1.27 (t, 6H, CH₃), 3.80 (s, 3H, OMe), 3.97-4.11 (m, 4H, OCH₂), 6.76 (br-s, 2H, NH₂), 6.80-6.83 (m, 1H), 7.82 (dd, 1H), 7.98 (dd, 1H)). Step 2: To a solution of methyl-4-amino-3-(diethoxyphosphoryl)benzoate (0.96 g, 3.15 mmol, 1 equiv) in THF: methanol: water (10 mL:2.5 mL, ratio:4:1:1) from Step 1 is added solid LiOH (0.45 g, 18.9 mmol, 6 equiv). After heating at 60° C. for 6 h, the reaction mixture is concentrate in vacuo, acidified to pH 2 and solid precipitated out. The solid is filtered and washed twice with 1N HCl to yield 4-amino-3-(diethoxyphosphoryl) benzoic acid ((0.49 mg, 57% yield). ¹H NMR (80 MHz, CDCl₃): δ=1.34 (t, 6H, CH₃), 3.85-4.38 (m, 4H, OCH₂), 5.74 (br-s, 2H, NH₂), 6.50-6.76 (m, 1H), 7.86-8.36 (m, 2H)). Step 3: To a solution of 4-amino-3-(diethoxyphosphoryl)benzoic acid (0.25 g, 0.92 mmol, 1 equiv) from Step 2 and PEG-amine (0.75 g, 1.01 mmol, 1.1 equiv) in MeOH (4.6 mL) is added 4-(4,6-dimethoxy-1,3,5-triazin-2-yl)-4-methylmorpholinium chloride (DMTMM) under Argon. After stirring at room temperature for 18h, the reaction mixture is concentrated in vacuo, and the residue is partitioned between DCM and brine. The organic layer was washed with 1N HCl, saturated sodium bicarbonate solution, dried with MgSO4, filtered and conc. in vacuo to yield PEG-amide ethyl phosphonate (0.35 g, 36% yield) which was subjected without purification to the next step. ¹H NMR (80 MHz, CD₃OD): δ=1.34 (t, 6H, CH₃), 3.44 (s, 3H, OCH3), 3.56-3.91 (m, PEG), 4.02-4.22 (m, 4H, OCH₂), 6.62-6.93 (m, 1H), 7.74-8.45 (m, 2H). Step 4: PEG-amide ethyl phosphonate (0.35 g, 0.36 mmol, 1 equiv) from Step 3 is suspended in 6 N HCl (aq.) (8 mL). After refluxing for 12 h, the reaction mixture is concentrated in vacuo. The residue is washed with MeOH, DCM and conc in vacuo to afford the PEG-amide phosphonic acid of compound (7k) ((0.31 g, 94% yield). ¹H NMR (80 MHz, D₂O): δ=3.02-4.06 (m, PEG), 7.36-7.52 (m, 1H), 7.99-8.09 (m, 1H)).

Compounds (7i) and (7j) can be prepared from similarly straightforward procedures. For example, compound (7i) can be prepared in 2-steps from 2-bromopyridin-3-amine (CAS Reg. #39856-58-1; Sigma-Aldrich, St. Louis, MO) as shown in the scheme below.

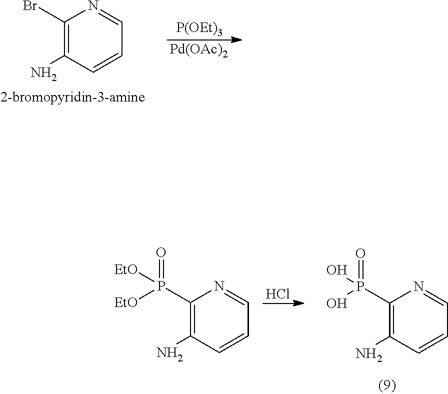

Compound (7j) is prepared similarly in 2-steps from 4-bromopyrimidin-5-amine (CAS Reg. #849353-34-0; Ambeed, Inc., Arlington Heights, IL, USA) as shown in the scheme below.

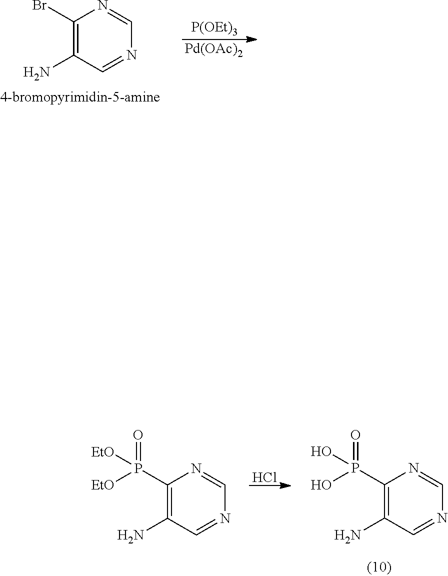 [SMILES: Nc1cncnc1Br ; CCOP(=O)(OCC)c1ncncc1N ; Cl ; Nc1cncnc1P(=O)(O)O ; "Pd(OAc)2" ; "P(OEt)3"]

The proline analog compounds (7m) and (7o) are prepared via a straightforward single step deprotection from commercially available protected precursor compounds. Accordingly, in at least one embodiment of the methods of the present disclosure that comprise the use of PFA, the method can further comprise contacting the sample with an unfixing agent capable of reversing PFA fixation, optionally, where the unfixing agent is selected from any one of compounds (7a)-(7o) shown in Table 2.

### D. Compositions of Bis-Carbamate Crosslinked Biomolecules

As shown by the reactions depicted in Schemes 2, 3, and 4, the use of the bis-imidazole carboxylate compounds of formula (I) in a method of fixing a biological sample results in the formation of bis-carbamate crosslinks between amine-bearing moieties of the biomolecules of the sample, e.g., proteins and nucleic acids. The bis-carbamate crosslinks formed between amine-bearing moieties of the biomolecule as a result of treatment with a compound of formula (I) can be represented by a structure of formula (Ia)

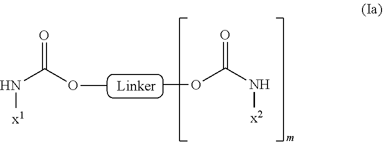

The structure of formula (Ia) depicts the amine-bearing moieties of the biomolecule as X¹ and X², and it is contemplated that X¹ and X² can be moieties on the same or on different biomolecules of the biological sample. The “Linker” can vary as it does in the compound of formula (I), comprising an ethylene glycol moiety, and/or a linear or branched alkane moiety of 2-24 carbons; and m is 1 to 12.

The bis-carbamate crosslinked biomolecules of formula (Ia) represent the “fixed” components of the fixed biological sample. As in other known fixation methods, it is believed that the covalent bis-carbamate crosslinks reduce and/or inhibit the ability of the biomolecule compounds of formula (Ia) to undergo the chemical processes resulting in degradation of the biological sample. Accordingly, the present disclosure also provides fixed biomolecules and compositions of such biomolecules that comprise the covalent bis-carbamate structure of formula (Ia), which is not found in the naturally occurring biomolecules. In at least one embodiment, the present disclosure provides a composition comprising a fixed biological sample, wherein the sample comprises crosslinks of formula (Ia), wherein, X¹ and X² are amine-bearing moieties of the same or different biomolecules of the sample; and “Linker” comprises an ethylene glycol moiety, and/or a linear or branched alkane moiety of 2-24 carbons; and m is 1 to 12.

Further, in at least one embodiment, the present disclosure also provides a composition comprising a fixed biological sample, wherein the sample comprises:
(a) a crosslinked biomolecule of formula (IIa)

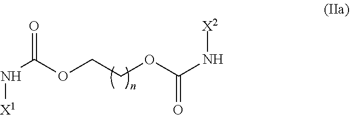

wherein n is 1 to 13;
(b) a crosslinked biomolecule of formula (IIIa)

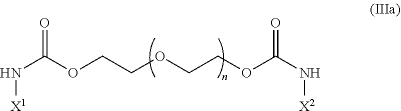

wherein, n is 1 to 12;
(c) a crosslinked biomolecule of formula (IVa)

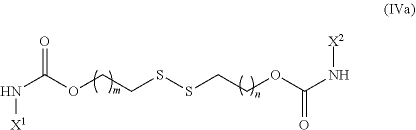

wherein m is 1 to 13, and n is 1 to 13;
(d) a crosslinked biomolecule of formula (Va)

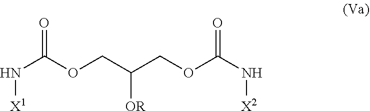

wherein R is selected from —H, —O(CO)—CH₃, and —O(CO)-imidazole; and/or
(e) a crosslinked biomolecule of formula (VIa)

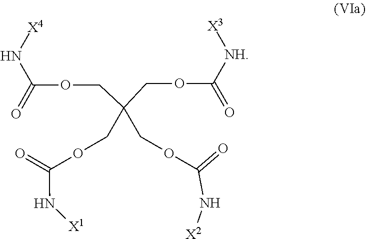

### E. Methods for Use of Unfixing agents

As shown in the above Scheme 2, the fixed biological samples prepared using the fixing reagents of the present disclosure (e.g., compound of formula (I)) comprise bis-carbamate crosslinks. The bis-carbamate crosslinks that act to fix the biomolecules in a biological molecule with a structure of formula (Ia) can be reversed with standard carbamate-cleaving compositions and methods. Thus, a further advantageous technical effect of the fixing reagent compositions and methods of present disclosure is their facile reversibility. The generalized un-fixing reaction that reverses the bis-carbamate crosslinks is shown in Scheme 5,

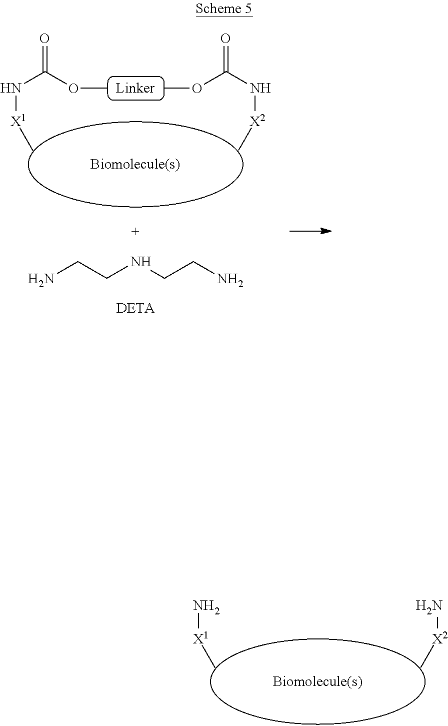

Accordingly, the methods for preparing a biological sample using a bis-imidazole carboxylate fixing reagent compound of formula (I) can further include a subsequent step of reversing the fixation by contacting the sample with unfixing agent. As shown in Scheme 5, the compound diethylenetriamine (“DETA”) is effective for the cleavage of carbamate bonds. See e.g., Noshita et al., “Diethylenetriamine-Mediated Direct Cleavage of Unactivated Carbamates and Ureas,” Org. Lett. 18:6062-6065 (2016). Similarly, the diamine compounds, including ethylenediamine (“EDA”), triethylenetetramine (“TETA”), and hydrazine monohydrate can cleave carbamate bonds. The carboxyesterase class of enzymes are capable of cleaving carbamate bonds, and it is contemplated that a carboxyesterase can also be used as an unfixing agent in a method of the present disclosure. Thus, in at least one embodiment, the present disclosure provides a method for preparing a biological sample by contacting the sample with a fixing reagent composition comprising a compound of formula (I), wherein the method further comprises contacting the sample with an unfixing agent, wherein the unfixing agent is a compound capable of cleaving a carbamate bond. In at least one embodiment that includes the unfixing agent, the compound capable of cleaving a carbamate bond is selected from DETA, EDA, TETA, hydrazine monohydrate, a carboxyesterase, or a combination thereof.

As illustrated by the fixing reagent compound of formula (IV), in some embodiments the “Linker” can include a disulfide bond. The addition of a disulfide to the bis-imidazole carboxylate fixing reagent compound allows for the formation of crosslinked biomolecules of formula (IVa). The presence of crosslinked biomolecules of formula (IVa) in a biological sample allows for an alternative two-step un-fixing reaction as illustrated in Scheme 6.

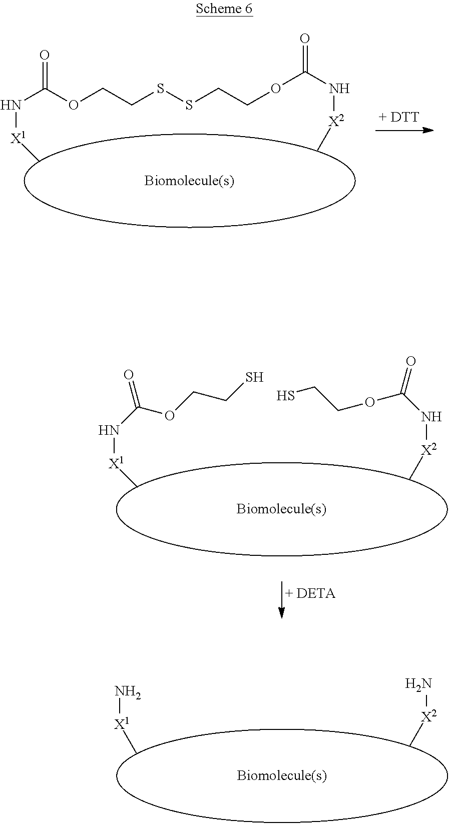

In the first step of the reaction of Scheme 6, the crosslinked biomolecule is treated with the well-known reagent, dithiothreitol (“DTT”) that results in the reductive cleavage of the disulfide bonds in the crosslinks. DTT treatment is commonly used and is easily carried out to completion under mild biological conditions. By cleaving the crosslinks, the DTT treatment un-fix the biomolecules to the extent that the crosslinks act to stabilize them from degradation. The resulting un-fixed biomolecules, however, still retain amine-bearing moieties that are modified with sulfhydryl-ethylene-carboxylate moieties. The carbamate-modified amine moieties can potentially interfere with certain subsequent assays of the biomolecules. For example, carbamate-modified nucleobases of nucleic acids will interfere with assays that utilize reverse transcription and amplification of nucleic acid sequences. Accordingly, in at least one embodiment, the present disclosure provides a method wherein a biological sample treated with composition comprising a fixation reagent compound of formula (IV) is contacted with a disulfide cleaving compound, such as DTT, and then contacted with a carbamate-cleaving compound, such as DETA. As shown in Scheme 6, following this two-step treatment, the bis-carbamate crosslinked amine-bearing moieties of the biomolecules are returned to their native state.

### F. Fixed Biological Samples in Partitions With Unfixing Agents

Recognized herein is the need for methods, compositions, kits, and systems for analyzing multiple cellular analytes (e.g., genomic, epigenomic, transcriptomic, metabolomic, and/or proteomic information) from fixed biological samples, e.g., individual cells, a population of cells, tissue samples, and other kinds of biological samples. The compositions and methods of the present disclosure are useful to prepare biological samples that are reversibly fixed with a fixing reagent compound of formula (I), then provided (or encapsulated) in discrete partitions along with an unfixing agent capable of reversing the fixed state of the biomolecules while sequestered in the partition. Accordingly, in some embodiments, the present disclosure provides a method for preparing a biological sample comprising: generating a discrete partition comprising (or encapsulating) a biological sample that has been fixed using a bis-imidazole carboxylate compound of formula (I), and an unfixing agent that is capable of reversing the bis-carbamate crosslinked molecules of formula (Ia). This method can further comprise an initial step of fixing the biological sample prior to generating the discrete partition.

The present disclosure provides methods, composition, kits, and systems for treating fixed biological samples in order to process cellular analytes. Cellular analytes that are suitable for use with the compositions and methods of the present disclosure include, without limitation, intracellular and partially intracellular analytes. The cellular analyte may be a protein, a metabolite, a metabolic byproduct, an antibody or antibody fragment, an enzyme, an antigen, a carbohydrate, a lipid, a macromolecule, or a combination thereof (e.g., proteoglycan) or other biomolecule. The cellular analyte may be a nucleic acid molecule. The cellular analyte may be a deoxyribonucleic acid (DNA) molecule or a ribonucleic acid (RNA) molecule. The DNA molecule may be a genomic DNA molecule. The cellular analyte may comprise coding or non-coding RNA. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (RNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA.

In some instances, the cellular analyte is associated with an intermediary entity, wherein the intermediary entity is analyzed to provide information about the cellular analyte and/or the intermediary entity itself. For instance, an intermediary entity (e.g., an antibody) may be bound to a partially intracellular analyte (e.g., a cell surface receptor), where the intermediary entity is processed to provide information about the intermediary entity, the partially intracellular analyte, or both. In one embodiment, the intermediary entity comprises an identifier (e.g., a barcode molecule) that can be used to generate barcode molecules (e.g., droplet-based barcoding) as further described herein.

The term “partition,” as used herein, generally, refers to a space or volume that may be suitable to contain one or more species or conduct one or more reactions. A partition may be a physical compartment, such as a droplet or well (e.g., a microwell). The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments

Methods, techniques, and protocols useful for partitioning biological samples (e.g., individual cells, biomolecular contents of cells, etc.) into discrete partitions (e.g., wells or droplets) are described in the art. In one embodiment, the discrete partitions generated act a nanoliter-scale container that can maintain separation of the partition contents from the contents of other partitions (e.g., droplets in the emulsion).

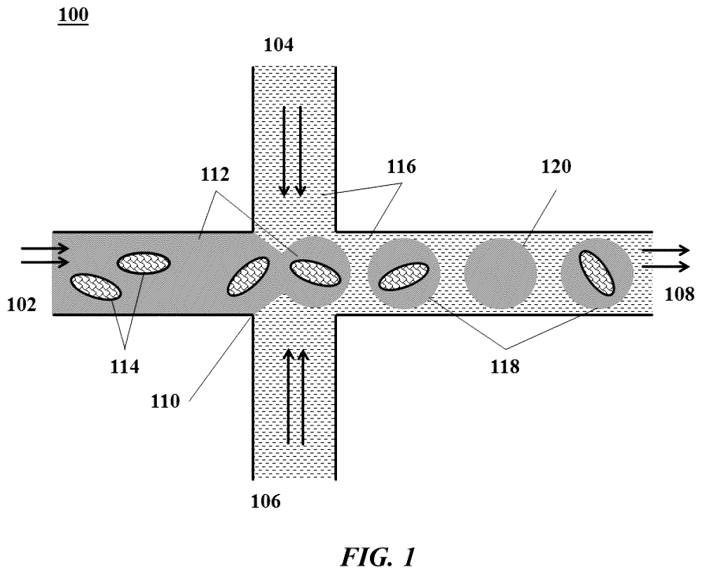

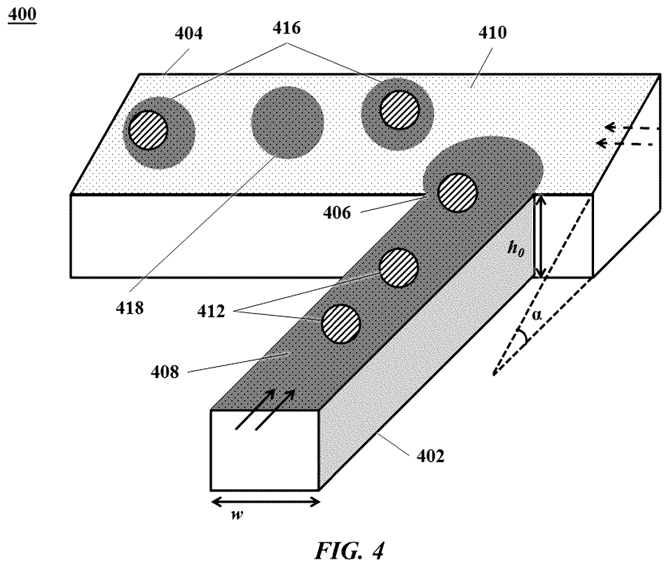

Methods and systems for creating stable discrete droplets comprising or encapsulating individual particles from biological samples in non-aqueous or oil emulsions are described in, e.g., U.S. Patent Application Publication Nos. 2010/0105112 and 2019/0100632, each of which is entirely incorporated herein by reference for all purposes. Briefly, discrete droplets in an emulsion comprising or encapsulating a biological sample is accomplished by introducing a flowing stream of an aqueous fluid containing the biological sample into a flowing stream of a non-aqueous fluid with which it is immiscible, such that droplets are generated at the junction of the two streams (see FIGS. 1-3). By providing the aqueous stream at a certain concentration and/or flow rate of the biological sample, the occupancy of the resulting droplets can be controlled. For example, the relative flow rates of the immiscible fluids can be selected such that, on average, the discrete droplet each contains less than one biological particle. Such a flow rate ensures that the droplets that are occupied are primarily occupied by a single sample (e.g., a single cell). Discrete droplets in an emulsion comprising or encapsulating a biological sample is also accomplished using a microfluidic architecture comprising a channel segment having a channel junction with a reservoir (see FIGS. 4-6).

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle.

In some cases, the droplets among a plurality of discrete droplets formed in the manner contain at most one particle (e.g., one support such as a bead) and one biological particle (e.g., one cell including one fixed cell or one un-fixed cell). The flows and microfluidic channel architectures also can be controlled to ensure a given number of singly occupied droplets, less than a certain level of unoccupied droplets, and/or less than a certain level of multiply occupied droplets.

In another aspect of the disclosure, fixed cells (e.g., cells fixed according to the fixing reagent compositions described herein) may then be partitioned (e.g., in a droplet or well) with other reagents for processing of one or more analytes as described herein. In one embodiment, the fixed cell may be co-partitioned with an unfixing agent. In one other embodiment, the partition comprising the fixed cell further comprises a support (e.g., a bead) comprising nucleic acid molecules suitable for barcoding of the one or more analytes. In another embodiment, the nucleic acid molecules may include nucleic acid sequences that provide identifying information, e.g., barcode sequence(s).

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, compositions, kits, and systems described herein, hybridization and reverse transcription of the nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

FIG. 1 shows an exemplary microfluidic channel structure 100 useful for generating discrete droplets comprising or encapsulating a particle from a biological sample, such as a single cell. The channel structure 100 can include channel segments 102, 104, 106 and 108 communicating at a channel junction 110. In operation, a first aqueous fluid 112 that that includes suspended particles (e.g., cells) from a biological sample 114 are transported along channel segment 102 into junction 110, while a second fluid 116 (or “partitioning fluid”) that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual particle from a biological sample 114 (such as droplet 118), or discrete droplet can be generated that includes more than one particle 114 (not shown in FIG. 1). A discrete droplet may contain no biological particle 114 (such as droplet 120). Each discrete droplet is capable of maintaining separation of its own contents (e.g., individual biological sample particle 114) from the contents of other droplets.

Typically, the second fluid 116 comprises an oil, such as a fluorinated oil, that includes a fluoro-surfactant that helps to stabilize the resulting droplets. Examples of useful partitioning fluids and fluoro-surfactants are described in e.g., U.S. Patent Application Publication No. 2010/0105112, which is entirely incorporated herein by reference for all purposes.

The microfluidic channels for generating discrete droplets as exemplified in FIG. 1 may be coupled to any of a variety of different fluid sources or receiving components, including reservoirs, tubing, manifolds, or fluidic components of other systems. Additionally, the microfluidic channel structure 100 may have other geometries, including geometries having more than one channel junction. For example, the microfluidic channel structure can have 2, 3, 4, or 5 channel segments each carrying biological sample particles, assay reagents, and/or beads that meet at a channel junction.

Generally, the fluids used in generating the discrete droplets are directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

One of ordinary skill will recognize that numerous different microfluidic channel designs are available that can be used with the methods and compositions of the present disclosure to provide discrete droplets containing a particle of a biological sample fixed with a compound of formula (I), an unfixing agent capable of cleaving bis-carbamate crosslinks (e.g., DETA), and/or a bead with a barcode and/or other assay reagents.

The inclusion of a barcode in a discrete partition (e.g., a well or a droplet) along with the biological sample provides a unique identifier that allows data from the biological sample to be distinguished and individually analyzed. Barcodes can be delivered previous to, subsequent to, or concurrent with the biological sample in discrete partition. For example, barcodes may be injected into droplets previous to, subsequent to, or concurrently with droplet generation. Barcodes useful in the methods and compositions of the present disclosure typically comprise a nucleic acid molecule (e.g., an oligonucleotide). The nucleic acid barcode molecules typically are delivered to a partition via a support, such as bead. In some cases, barcode nucleic acid molecules are initially associated with the bead upon providing the discrete partition (e.g., providing a discrete well or upon generation of the discrete droplet), and then released from the bead upon application of a stimulus to the partition (e.g., the well or the droplet). Barcode carrying beads useful in the methods and compositions of the present disclosure are described in further detail elsewhere herein.

Methods and systems for partitioning barcode carrying beads into droplets are provided in U.S. Pat. Nos. 10,480,029, 10,858,702, and 10,725,027, US. Patent Publication Nos. 2019/0367997 and 2019/0064173, and International Application Nos. PCT/US20/17785 and PCT/US20/020486, each of which is herein entirely incorporated by reference for all purposes.

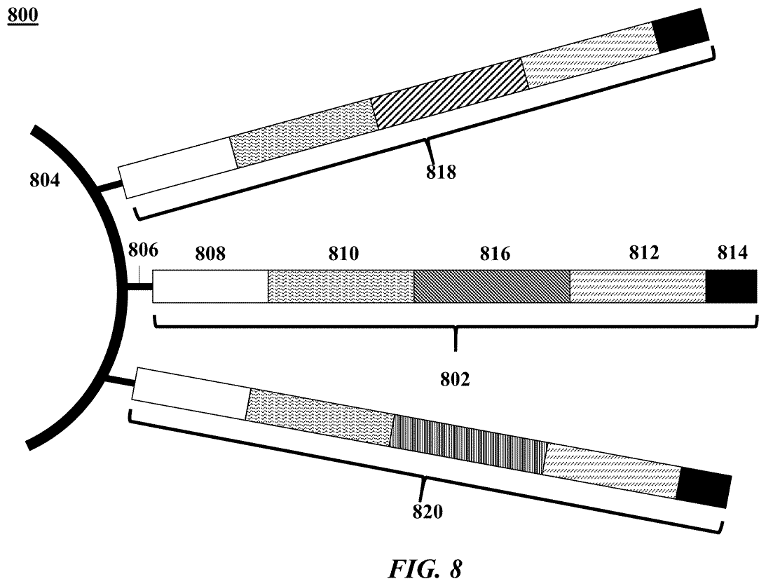

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, antibody, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

A biological particle (e.g., cell, fixed cell, un-fixed cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of a mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810.

However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., a cell, a fixed cell, an un-fixed cell, etc.). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing). The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

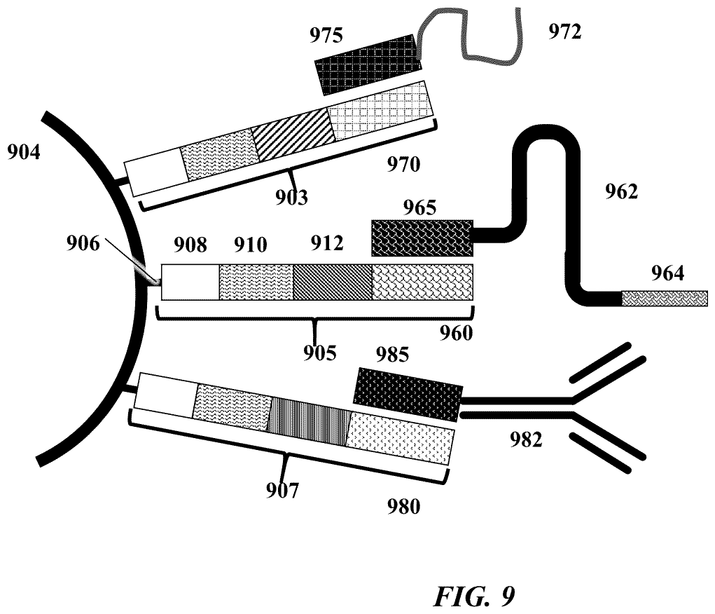

FIG. 9 illustrates another example of a barcode carrying bead. A nucleic acid molecule 905, such as an oligonucleotide, can be coupled to a bead 904 by a releasable linkage 906, such as, for example, a disulfide linker. The nucleic acid molecule 905 may comprise a first capture sequence 960. The same bead 904 may be coupled (e.g., via releasable linkage) to one or more other nucleic acid molecules 903, 907 comprising other capture sequences. The nucleic acid molecule 905 may be or comprise a barcode. As noted elsewhere herein, the structure of the barcode may comprise a number of sequence elements, such as a functional sequence 908 (e.g., flow cell attachment sequence, sequencing primer sequence, etc.), a barcode sequence 910 (e.g., bead-specific sequence common to bead, partition-specific sequence common to partition, etc.), and a unique molecular identifier 912 (e.g., unique sequence within different molecules attached to the bead), or partial sequences thereof. The capture sequence 960 may be configured to attach to a corresponding capture sequence 965. In some instances, the corresponding capture sequence 965 may be coupled to another molecule that may be an analyte or an intermediary carrier. For example, as illustrated in FIG. 9, the corresponding capture sequence 965 is coupled to a guide RNA molecule 962 comprising a target sequence 964, wherein the target sequence 964 is configured to attach to the analyte. Another oligonucleotide molecule 907 attached to the bead 904 comprises a second capture sequence 980 which is configured to attach to a second corresponding capture sequence 985. As illustrated in FIG. 9, the second corresponding capture sequence 985 is coupled to an antibody 982. In some cases, the antibody 982 may have binding specificity to an analyte (e.g., surface protein). Alternatively, the antibody 982 may not have binding specificity. Another oligonucleotide molecule 903 attached to the bead 904 comprises a third capture sequence 970 which is configured to attach to a second corresponding capture sequence 975. As illustrated in FIG. 9, the third corresponding capture sequence 975 is coupled to a molecule 972. The molecule 972 may or may not be configured to target an analyte. The other oligonucleotide molecules 903, 907 may comprise the other sequences (e.g., functional sequence, barcode sequence, UMI, etc.) described with respect to oligonucleotide molecule 905. While a single oligonucleotide molecule comprising each capture sequence is illustrated in FIG. 9, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 904 may comprise other capture sequences. Alternatively, or in addition, the bead 904 may comprise fewer types of capture sequences (e.g., two capture sequences). Alternatively or in addition, the bead 904 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

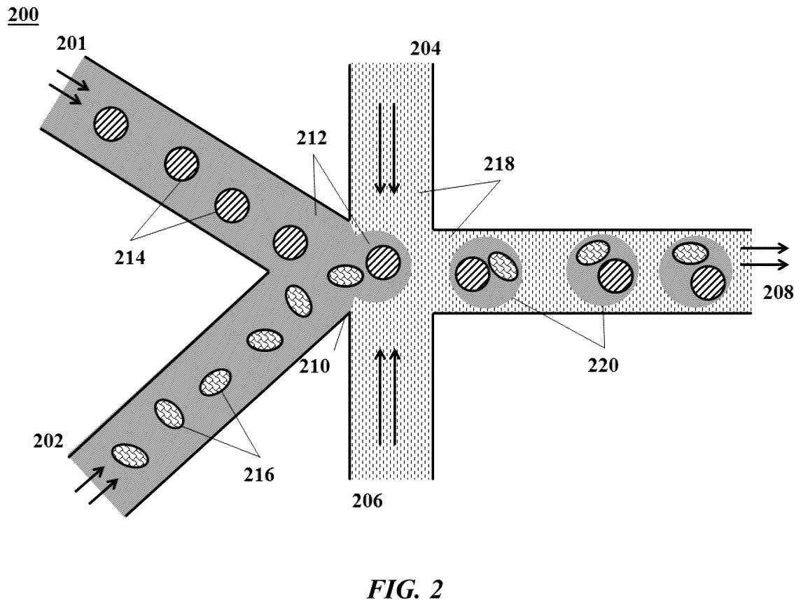

FIG. 2 shows an exemplary microfluidic channel structure 200 for generating discrete droplets comprising or encapsulating a barcode carrying bead 214 along with a biological sample particle 216. The channel structure 200 includes channel segments 201, 202, 204, 206 and 208 in fluid communication at a channel junction 210. In operation, the channel segment 201 transports an aqueous fluid 212 that can include a plurality of beads 214 (e.g., beads carrying barcode oligonucleotides) along the channel segment 201 into junction 210. The plurality of beads 214 may be sourced from a suspension of beads. For example, the channel segment 201 can be connected to a reservoir comprising an aqueous suspension of beads 214. The channel segment 202 transports the aqueous fluid 212 that includes a plurality of biological sample particles 216 along the channel segment 202 into junction 210. The plurality of biological sample particles 216 may be sourced from a suspension of biological sample particles. For example, the channel segment 202 may be connected to a reservoir comprising an aqueous suspension of biological sample particles 216. In some instances, the aqueous fluid 212 in either the first channel segment 201 or the second channel segment 202, or in both segments, can include one or more reagents, as further described elsewhere herein. For example, in some embodiments of the present disclosure, where the biological sample particles are fixed with a compound of formula (I), the aqueous fluid in the first and/or second channel segments that delivers the biological sample and beads, respectively, can include an unfixing agent capable of cleaving bis-carbamate crosslinks (e.g., DETA). The second fluid 218 that is immiscible with the aqueous fluid 212 is delivered to the junction 210 from each of channel segments 204 and 206. Upon meeting of the aqueous fluid 212 from each of channel segments 201 and 202 and the second fluid 218 (e.g., a fluorinated oil) from each of channel segments 204 and 206 at the channel junction 210, the aqueous fluid 212 is partitioned into discrete droplets 220 in the second fluid 218 and flow away from the junction 210 along channel segment 208. The channel segment 208 can then deliver the discrete droplets comprising or encapsulating the biological sample particle and barcode carrying bead to an outlet reservoir fluidly coupled to the channel segment 208, where they can be collected.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Using such a channel system as exemplified in FIG. 2, discrete droplets 220 can be generated that comprise or encapsulate an individual biological particle of a biological sample, and one bead, wherein the bead can carry a barcode and/or another reagent. It is also contemplated, that in some instances, a discrete droplet may be generated using the channel system of FIG. 2, wherein droplet includes more than one individual biological sample particle or includes no biological sample. Similarly, in some embodiments, the discrete droplet may include more than one bead or no bead. A discrete droplet also may be completely unoccupied (e.g., no bead or biological sample).

In some embodiments, it is desired that the beads, biological sample particles, and generated discrete droplets flow along channels at substantially regular flow rates that generate a discrete droplet containing a single bead and a single biological sample particle. Regular flow rates and devices that may be used to provide such regular flow rates are known in the art, see e.g., U.S. Patent Publication No. 2015/0292988, which is hereby incorporated by reference herein in its entirety. In some embodiments, the flow rates are set to provide discrete droplets containing a single bead and a biological sample particle with a yield rate of greater than 5%, 10%, 20%, 30%, 40%, 50%, 60%, 70%, 80%, 90%, or 95%.

### G. Uses of Supports Provided in Partitions

Supports that can carry barcodes and/or other reagents that are useful with the compositions and methods of the present disclosure and can include, without limitation, beads that are porous, non-porous, solid, semi-solid, semi-fluidic, fluidic, and/or a combination thereof. In some embodiments, the bead can be made of a material that is dissolvable, disruptable, and/or degradable, such as a gel bead comprising a hydrogel. Alternatively, in some embodiments, the bead is not degradable.

In some embodiments of the present disclosure, the bead is provided in a discrete partition (e.g., the bead is provided or encapsulated in a discrete droplet or provided in a discrete well) with a biological sample is a bead. Typically, the bead useful in the embodiments disclosed herein comprise a hydrogel. Such gel beads can be formed from molecular precursors, such as a polymeric or monomeric species, that undergo a reaction to form crosslinked gel polymer. Another semi-solid bead useful in the present disclosure is a liposomal bead. In some embodiments, beads used can be solid beads that comprise a metal including iron oxide, gold, and silver. In some cases, the bead may be a silica bead. In some cases, the bead can be rigid. In other cases, the bead may be flexible and/or compressible. Generally, the beads can be of any suitable shape. Examples of bead shapes include, but are not limited to, spherical, non-spherical, oval, oblong, amorphous, circular, cylindrical, and variations thereof.

The plurality beads used in the embodiments can be of uniform size or they can comprise a collection of heterogeneous sizes. In some cases, the diameter of a bead is at least about 1 micron (μm), 5 μm, 10 μm, 20 μm, 30 μm, 40 μm, 50 μm, 60 μm, 70 μm, 80 μm, 90 μm, 100 μm, 250 μm, 500 μm, 1000 μm (1 mm), or greater. In some cases, a bead may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In some embodiments, the beads used are a population or plurality of beads having a relatively monodisperse size distribution. Typically, where it is desirable to provide a consistent amount of reagent within a discrete partition (e.g., a well or a droplet), the use of relatively consistent bead characteristics, such as size, provides overall consistency in the content of each partition. For example, the beads useful in the embodiments of the present disclosure can have size distributions that have a coefficient of variation in their cross-sectional dimensions of less than 50%, less than 40%, less than 30%, less than 20%, and in some cases less than 15%, less than 10%, less than 5%, or less.

The beads useful in the methods and compositions of the present disclosure can comprise a range of natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

Although FIG. 1 and FIG. 2 have been described in terms of providing substantially singly occupied discrete droplets, it is also contemplated in certain embodiments that it is desirable to provide multiply occupied discrete droplets, e.g., a single droplet that contains two, three, four or more cells from a biological sample, and/or multiple different beads, such as a bead carrying a barcode nucleic acid molecule and/or a bead carrying a reagent such as a lysis agent, an unfixing agent and/or assay reagent. Accordingly, as noted elsewhere herein, the flow characteristics of the biological particle and/or the beads can be controlled to provide for such multiply occupied droplets. In particular, the flow parameters of the liquids used in the channel structures may be controlled to provide a given droplet occupancy rate greater than about 50%, greater than about 75%, and in some cases greater than about 80%, 90%, 95%, or higher.

In some embodiments, the beads useful in the compositions and methods of the present disclosure are beads capable of delivering reagents (e.g., an unfixing agent, and/or an assay reagent) into the discrete partition (e.g., a droplet) containing the biological sample particle that has been fixed by treatment with a compound of formula (I). In some embodiments, the different beads (e.g., containing different reagents) can be introduced from different sources into different inlets leading to a common droplet generation junction (e.g., junction 210). In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

The discrete droplets described herein generally comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. In some embodiments, the discrete droplets generated that comprise or encapsulate a biological particle from a sample have overall volumes that are less than about 1000 pL, 900 pL, 800 pL, 700 pL, 600 pL, 500 pL, 400pL, 300 pL, 200 pL, 100pL, 50 pL, 20 pL, 10 pL, 1 pL, or less. It will be appreciated that the sample fluid volume, e.g., including co-partitioned biological particles and/or beads, within the droplets may be less than about 90% of the above described volumes, less than about 80%, less than about 70%, less than about 60%, less than about 50%, less than about 40%, less than about 30%, less than about 20%, or less than about 10% of the above described volumes.

The methods of generating discrete droplets useful with the compositions and methods of the present disclosure, result in the generation of a population or plurality of discrete droplets containing a biological sample particle (e.g., a biological sample fixed treatment with a compound of formula (I)) and other reagents (e.g., an unfixing agent, such as DETA). Generally, the methods are easily controlled to provide for any suitable number of droplets. For example, at least about 1,000 discrete droplets, at least about 5,000 discrete droplets, at least about 10,000 discrete droplets, at least about 50,000 discrete droplets, at least about 100,000 discrete droplets, at least about 500,000 discrete droplets, at least about 1,000,000 discrete droplets, at least about 5,000,000 discrete droplets, at least about 10,000,000 discrete droplets, or more discrete droplets can be generated or otherwise provided. Moreover, the plurality of discrete droplets may comprise both unoccupied and occupied droplets.

As described elsewhere herein, in some embodiments of the compositions and methods of the present disclosure, the generated discrete droplets comprising or encapsulating a biological sample particle, and optionally, one or more different beads, also contain other reagents. In some embodiments, the other reagents contained in or encapsulated in the droplet include lysis and/or unfixing agents that act to release and/or un-fix the biomolecule contents of the biological sample particle within the droplet. In some embodiments, the lysis and/or unfixing agents can be contacted with the biological sample suspension concurrently with, or immediately prior to, the introduction of the biological sample particles into the droplet generation junction of the microfluidic system (e.g., junction 210). In some embodiments, the agents are introduced through an additional channel or channels upstream of the channel junction.

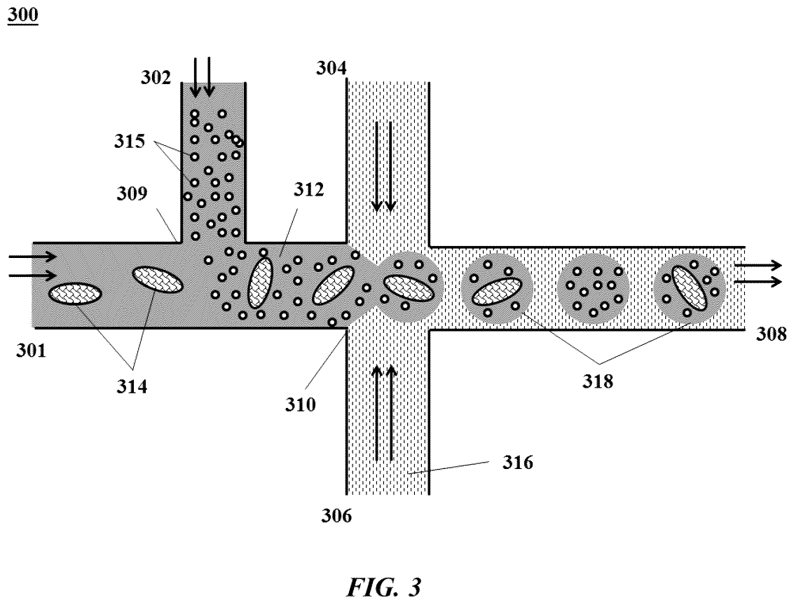

In some embodiments, a biological sample particle can be co-partitioned along with the other reagents. FIG. 3 shows an example of a microfluidic channel structure 300 for co-partitioning biological sample particles and other reagents, including lysis and/or unfixing agents. The channel structure 300 can include channel segments 301, 302, 304, 306 and 308. Channel segments 301 and 302 communicate at a first channel junction 309. Channel segments 302, 304, 306, and 308 communicate at a second channel junction 310. In exemplary co-partitioning operation, the channel segment 301 may transport an aqueous fluid 312 that includes a plurality of biological sample particles 314 (e.g., a fixed biological sample) along the channel segment 301 into the second junction 310. As an alternative or in addition to, channel segment 301 may transport beads (e.g., gel beads that carry barcodes). For example, the channel segment 301 may be connected to a reservoir comprising an aqueous suspension of biological sample particles 314. Upstream of, and immediately prior to reaching, the second junction 310, the channel segment 301 may meet the channel segment 302 at the first junction 309. The channel segment 302 can transport a plurality of reagents 315 (e.g., lysis or unfixing agents) in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological sample particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., a fluorinated oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 is partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be collected for further analysis.

Discrete droplets generated can include an individual biological sample particle 314 and/or one or more reagents 315, depending on what reagents are included in channel segment 302. In some instances, a discrete droplet generated may also include a barcode carrying bead (not shown), such as can be added via other channel structures described elsewhere herein. In some instances, a discrete droplet may be unoccupied (e.g., no reagents, no biological particles). Generally, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological sample particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, a, etc.) of the channel structure 400. A plurality of droplets can be collected in the reservoir 404 by continuously injecting the aqueous fluid 408 from the channel segment 402 through the junction 406.

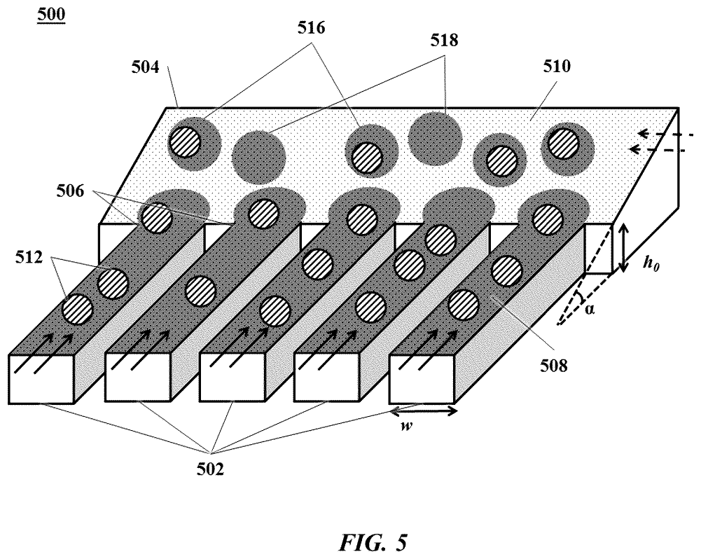

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

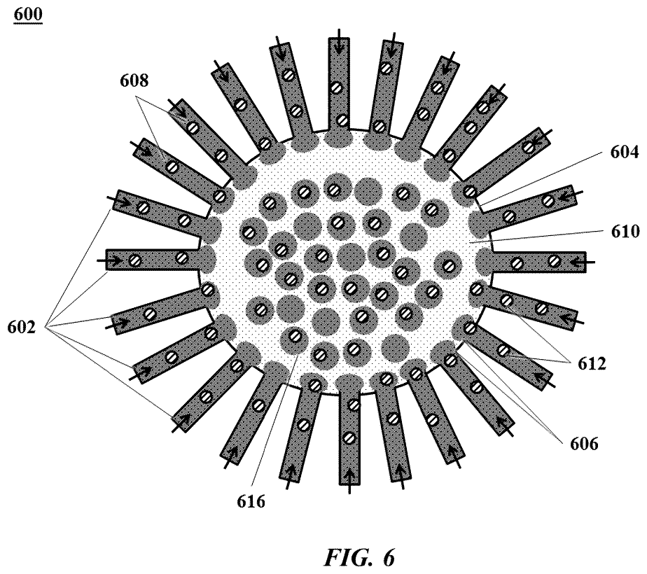

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 4 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof. Additional aspects of such microfluidic structures, including systems and methods implementing the same, are provided in US Published patent application No 20190323088, which is incorporated herein by reference in its entirety.

Once the lysis and/or unfixing agents are co-partitioned in a partition (e.g., a well or a droplet) with a particle of a biological sample fixed with a compound of formula (I), these reagents can facilitate the release and un-fixing of the biomolecular contents of the biological sample particle within the partition. As described elsewhere herein, the un-fixed biomolecular contents released in a partition remain discrete from the contents of other partitions, thereby allowing for detection and quantitation of the biomolecular analytes of interest present in that distinct biological sample.

Examples of lysis agents useful in the compositions and methods of the present disclosure include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological samples' contents into the partition (e.g., the well or the droplet). For example, in some cases, surfactant-based lysis solutions may be used to lyse cells, although these may be less desirable for emulsion based systems where the surfactants can interfere with stable emulsions. In some embodiment, the lysis solutions can include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as the provision or encapsulation of biological particles that may be in addition to or in place of droplet partitioning, where any pore size of the encapsulating material is sufficiently small to retain nucleic acid fragments of a given size, following cellular disruption.

In addition to the lysis and/or unfixing agents co-partitioned into discrete partitions (e.g., wells or droplets) with the biological sample particles, it is further contemplated that other assay reagents can also be co-partitioned in the partition. For example, DNase and Rnase inactivating agents or inhibitors, such as proteinase K, chelating agents, such as EDTA, and other reagents employed in removing or otherwise reducing negative activity or impact of different cell lysate components on subsequent processing of nucleic acids.

In some embodiments, the biological sample particles provided in or encapsulated in discrete partitions (e.g., wells or droplets) with other reagents are exposed to an appropriate stimulus to release the biomolecular contents of the sample particles and/or the contents of a co-partitioned bead. For example, in some embodiments, a chemical stimulus may be co-partitioned in the partition along with a biological sample particle and a bead (e.g., a gel bead) to allow for the degradation of the bead and release of its contents into the partition. In some embodiments, a discrete partition can be provided (e.g., a droplet can be generated) along with a particle of a biological sample fixed with a compound of formula (I) and an unfixing agent capable of cleaving bis-carbamate crosslinks (e.g., DETA), wherein the unfixing agent is contained in a bead (e.g., a gel bead) that can be degraded by heat stimulus. In such an embodiment, the partition is exposed to heat stimulus thereby degrading the bead and releasing the unfixing agent. In another embodiment, it is contemplated that a partition containing (e.g., a droplet providing or encapsulating) a particle of a biological sample fixed with a compound of formula (I), and two different beads (e.g., one bead carrying an unfixing agent, and one bead carrying assay reagents), wherein the contents of the two different beads are released by non-overlapping stimuli (e.g., a chemical stimulus and a heat stimulus). Such an embodiment can allow the release of the different reagents into the same discrete partition at different times. For example, a first bead, triggered by heat stimulus, releases an unfixing agent into the partition, and then after a set time, a second bead, triggered by a chemical stimulus, releases assay reagents that detect analytes of the biological sample particle that has been un-fixed by contact the unfixing agent.

Additional assay reagents may also be co-partitioned into discrete partitions (e.g., wells or droplets) with the biological samples, such as endonucleases to fragment a biological sample's DNA, DNA polymerase enzymes and dNTPs used to amplify the biological sample's nucleic acid fragments and to attach the barcode molecular tags to the amplified fragments. Other enzymes may be co-partitioned, including without limitation, polymerase, transposase, ligase, proteinase K, Dnase, etc. Additional assay reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some embodiments, template switching can be used to increase the length of cDNA generated in an assay. In some embodiments, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner.

Once the contents of a biological sample cell are released into a discrete partition (e.g., a well or a droplet), the biomolecular components (e.g., macromolecular constituents of biological samples, such as RNA, DNA, or proteins) contained therein may be further processed within the partition. In accordance with the methods and systems described herein, the biomolecular contents of individual biological samples can be provided with unique barcode identifiers, and upon characterization of the biomolecular components (e.g., in a sequencing assay) they may be attributed as having been derived from the same biological sample. The ability to attribute characteristics to individual biological samples or groups of biological samples is provided by the assignment of a nucleic acid barcode sequence specifically to an individual biological sample or groups of biological samples.

In some embodiments, the unique identifier barcodes are provided in the form of nucleic acid molecules (e.g., oligonucleotides) that comprise sequences that may be attached to or otherwise associated with the nucleic acid contents of individual biological sample, or to other components of the biological sample, and particularly to fragments of those nucleic acids. In some embodiments, only one nucleic acid barcode sequence is associated with a given discrete partition (e.g., a well or a droplet), although in some cases, two or more different barcode sequences may be present. The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

In some embodiments, the nucleic acid barcode molecules can also comprise other functional sequences useful in the processing of the nucleic acids from the biological sample in the partition (e.g., the well or the droplet). These functional sequences can include, e.g., targeted or random/universal amplification primer sequences for amplifying the nucleic acid molecules from the individual biological samples within the partitions while attaching the associated barcode sequences, sequencing primers or primer recognition sites, hybridization or probing sequences, e.g., for identification of presence of the sequences or for pulling down barcoded nucleic acid molecules, or any of a number of other potential functional sequences.

In some embodiments, large numbers of nucleic acid barcode molecules (e.g., oligonucleotides) are releasably attached to beads, wherein all of the nucleic acid molecules attached to a particular bead will include the same nucleic acid barcode sequence, but where a large number of diverse barcode sequences are represented across the population of beads used. In some embodiments, gel beads (e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

The nucleic acid barcode molecules can be released from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for provision or encapsulation of biological samples and may be degraded for release of the attached nucleic acid molecules through exposure to a reducing agent, such as DTT.

### H. Use of Fixed Biological Samples and Unfixing Agents in Partition-Based Assays

As disclosed elsewhere herein, the compositions and methods of the present disclosure allow for the preparation of a biological sample that has been fixed with a bis-imidazole-carboxylate compound of formula (I) to be provided in a discrete partition (e.g., provided in a discrete well, or provided or encapsulated in a discrete droplet (optionally, as a single cell)), optionally, together with a lysis agent and/or an unfixing agent that is capable of reversing bis-carbamate crosslinks of the sample, and thereby allowing the cellular analytes of the sample to be assayed as if they were obtained from a fresh biological sample. In one embodiment, a single cell (e.g., a single fixed cell) is provided in a discrete partition. The fixing reagent compounds, methods, and associated unfixing agents of the present disclosure allow for a fresh biological sample to be immediately preserved, and then stored for a period of time before it is provided in a partition (e.g., provided in a well, or provided in or encapsulated in a droplet) with a lysis agent and/or an unfixing agent. Typically, other materials, such as a unique nucleic acid barcode molecule and assay reagents are also provided in the partition (e.g., provided in the well, or provided or encapsulated in the droplet). Accordingly, it is contemplated that the methods of the present disclosure can be carried out wherein the amount of time between the fixation of the biological sample with a bis-imidazole-carboxylate compound of formula (I) and providing the sample in a discrete partition (e.g., in a well, in a droplet, or by encapsulation in a discrete droplet) for processing is at least 1 hour, at least 2 hours, at least 6 hours, at least 12 hours, at least 24 hours, at least 72 hours, at least 1 week, at least 1 month, at least 6 months, or longer.

Generally, it is contemplated that any of the methods for preparing a biological sample using a fixing reagent composition comprising a compounds of formula (I) (including compounds of formulas (II), (III), (IV), (V), or compound (6)) disclosed herein can also be used to prepare a fixed biological sample that is provided in a partition (e.g., provided in a well or encapsulated in a droplet), optionally, together with a lysis agent and/or an unfixing agent. Similarly, it is contemplated that any of the compositions disclosed herein comprising bis-carbamate crosslinked biomolecules of formula (Ia) (including compounds of formulas (Iia), (IIIa), (Iva), (Va), or (Via)) can be provided in a partition (e.g., provided in a well or encapsulated in a droplet) with an unfixing agent and used in a partition-based assay method (e.g., a well- or a droplet-based assay method). Accordingly, in at least one embodiment, the present disclosure provides an assay method comprising:
(a) generating a discrete partition comprising a fixed biological sample (a well comprising or a droplet comprising or encapsulating a fixed biological sample), a lysis and/or an unfixing agent, and assay reagents, wherein the fixed sample comprises a crosslinked biomolecule of formula (Ia)

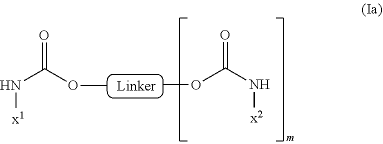

wherein, X¹ and X² are amine-bearing moieties of the same or different biomolecules of the sample; “Linker” comprises an ethylene glycol moiety, and/or a linear or branched alkane moiety of 2-24 carbons; and m is 1 to 12; the unfixing agent comprises a compound capable of cleaving a carbamate bond; optionally, wherein the compound capable of cleaving a carbamate bond selected from DETA, EDA, hydrazine monohydrate, a carboxyesterase, or a combination thereof; and
(b) detecting analytes from the reaction of the assay reagents and the un-fixed biological sample.

Optionally, the steps of the assay method can further comprise preparing the biological sample by contacting the sample with a fixing reagent composition comprising a compound of formula (I) prior to generating the discrete partition (e.g., the well or the droplet).

A wide range of droplet-based assays and systems are known in the art. Assays and systems that are suitable for use with the compositions and methods of the present disclosure include, without limitation, those described in U.S. Pat. Nos. 9,694,361, 10,357,771, 10,273,541, and 10,011,872, as well as US Published patents application Nos. 20180105808, 20190367982, and 20190338353, each of which is incorporated herein by reference in its entirety. It is contemplated that any assay that can be carried out using a fresh biological sample, such as a single cell provided in or encapsulated in a droplet with a bead carrying a barcode, can also be carried out using a fixed biological sample, prepared using the fixing reagents, and associated methods of the present disclosure. That is, in any droplet-based assay using a fresh biological sample, the droplet-based assay protocol can also be carried out wherein the fresh biological sample is fixed prior to running the assay protocol. In such an assay the protocol can comprise providing or encapsulating the fixed biological sample in a discrete droplet together with a lysis agent and/or an unfixing agent and assay reagents.

In some embodiments of the assay methods, the discrete partition (e.g., a well or a droplet) further comprises one or more beads. In some embodiments, the bead(s) can contain the assay reagents and/or the unfixing agent. In some embodiments, a barcode is carried by or contained in a bead. Compositions, methods and systems for sample preparation, amplification, and sequencing of biomolecules from single cells encapsulated with barcodes in droplets are provided in e.g., US Pat. Publication No. 20180216162A1, which is hereby incorporated by reference herein.

Assay reagents can include those used to perform one or more additional chemical or biochemical operations on a biological sample provided or encapsulated in a partition (e.g., in a well, in a droplet, or encapsulated in a droplet). Accordingly, assay reagents useful in the assay method include any reagents useful in performing a reaction such as nucleic acid modification (e.g., ligation, digestion, methylation, random mutagenesis, bisulfite conversion, uracil hydrolysis, nucleic acid repair, capping, or decapping), nucleic acid amplification (e.g., isothermal amplification or PCR), nucleic acid insertion or cleavage (e.g., via CRISPR/Cas9-mediated or transposon-mediated insertion or cleavage), and/or reverse transcription. Additionally, useful assay reagents can include those that allow the preparation of a target sequence or sequencing reads that are specific to the macromolecular constituents of interest at a higher rate than to non-target sequence specific reads.

In addition, the present disclosure provides compositions and systems related to the analysis of fixed biological samples. In one embodiment, the present disclosure provides a composition comprising a plurality of partitions, wherein a subset of said plurality of partitions comprises cells fixed using a fixing reagent composition and/or associated methods described herein. The partitions may further comprise a lysis agent and/or an unfixing agent. In another embodiment, a partition of the plurality of partitions comprises a fixed cell and a lysis agent and/or an unfixing agent. In certain embodiments, the fixed cell is a single fixed cell. In other embodiments the present disclosure provides a composition comprising a partition, wherein the partition comprises a fixed cell, prepared with a fixing reagent of the present disclosure, a lysis agent and/or an unfixing agent, as described herein. The partition may be a droplet or a well. In another embodiment, the partition can further comprise a protease. In another embodiment, the partition or partitions described herein comprising a fixed cell may further comprise assay reagents, optionally where the assay reagents comprise one or more of the following: a reverse transcriptase, a bead, and reagents for a nucleic acid extension reaction. In an additional embodiment, the compositions of the present disclosure have or are provided at a temperature other than ambient temperature or non-ambient temperature. In one embodiment, the temperature is below ambient temperature or above ambient temperature.

As described elsewhere herein, partitioning approaches may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions. For example, an occupied partition according the present disclosure comprises a fixed cell (e.g., fixed according to a fixing reagent composition as described herein) and an unfixing agent.

In another aspect, the present disclosure concerns methods and compositions for the partitioning of a plurality of fixed cells (e.g., fixed according to a fixing reagent composition as described herein) into individual partitions. In some cases, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, about 10,000,about 15,000, about 20,000, about 25,000, about 30,000, about 35,000, about 40,000, about 50,000, about 60,000, about 70,000, about 80,000, about 90,000 or about 100,000 fixed cells may be partitioned into individual partitions. In some instances, the method further comprises partitioning about 50 to about 20,000 fixed cells with each of a plurality of supports comprising the adaptor comprising the barcode sequence, wherein the barcode sequence is unique among each of the plurality of supports.

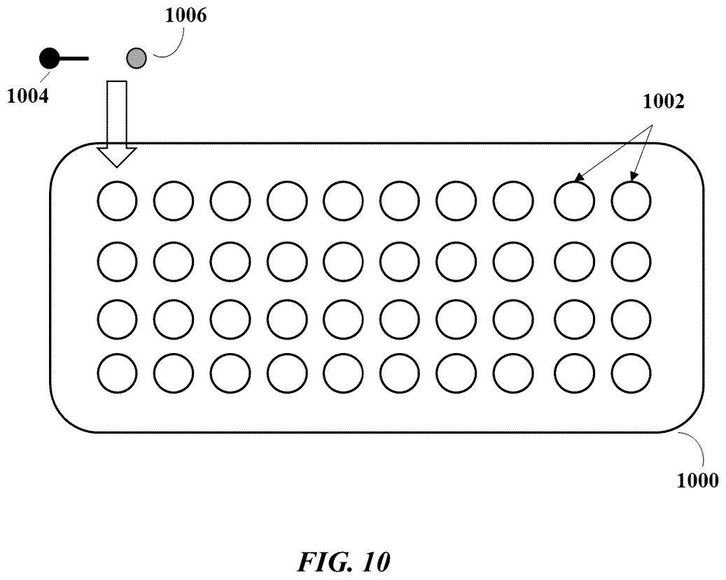

FIG. 10 schematically illustrates an example of a microwell array. The array can be contained within a substrate 1000. The substrate 1000 comprises a plurality of wells 1002. The wells 1002 may be of any size or shape, and the spacing between the wells, the number of wells per substrate, as well as the density of the wells on the substrate 1000 can be modified, depending on the particular application. In one such example application, a sample molecule 1006, which may comprise a cell (e.g., a fixed cell or an un-fixed cell) or cellular components (e.g., nucleic acid molecules) is co-partitioned with a bead 1004, which may comprise a nucleic acid barcode molecule coupled thereto. The wells 1002 may be loaded using gravity or other loading technique (e.g., centrifugation, liquid handler, acoustic loading, optoelectronic, etc.). In some instances, at least one of the wells 1002 contains a single sample molecule 1006 (e.g., cell) and a single bead 1004.

Reagents may be loaded into a well either sequentially or concurrently. In some cases, reagents are introduced to the device either before or after a particular operation. In some cases, reagents (which may be provided, in certain instances, in droplets or beads) are introduced sequentially such that different reactions or operations occur at different steps. The reagents (or droplets or beads) may also be loaded at operations interspersed with a reaction or operation step. For example, droplets or beads comprising reagents for fragmenting polynucleotides (e.g., restriction enzymes) and/or other enzymes (e.g., transposases, ligases, polymerases, etc.) may be loaded into the well or plurality of wells, followed by loading of droplets or beads comprising reagents for attaching nucleic acid barcode molecules to a sample nucleic acid molecule. Reagents may be provided concurrently or sequentially with a sample, such as a cell (e.g., a fixed cell or an un-fixed cell) or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead or droplet. These beads or droplets may be loaded into a partition (e.g., a microwell) before, after, or concurrently with the loading of a cell (e.g., a fixed cell or an un-fixed cell), such that each cell is contacted with a different bead or droplet. This technique may be used to attach a unique nucleic acid barcode molecule to nucleic acid molecules obtained from each cell (e.g., a fixed cell or an un-fixed cell). Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell, e.g., a single fixed cell or a single un-fixed cell) and a single bead (such as those described herein, which may, in some instances, also be provided or encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where cells, e.g., fixed cells or un-fixed cells are loaded in the microwells, one of the droplets may comprise reagents for further processing, e.g., lysis reagents for lysing the cell, upon droplet merging.

A droplet or support (e.g., a bead) may be partitioned into a well. The droplets may be selected or subjected to pre-processing prior to loading into a well. For instance, the droplets may comprise cells, e.g., fixed cells or un-fixed cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell (e.g., a fixed cell or an un-fixed cell) distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell or cellular components) or derivatives thereof. Characterization techniques such as microscopy or imaging may be useful in measuring sample profiles in fixed spatial locations. For instance, when cells (e.g., fixed cells or un-fixed cells) are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, cell-cell interactions (when two or more cells are co-partitioned). Alternatively or in addition to, imaging may be used to characterize a quantity of amplification products in the well.

In operation, a well may be loaded with a sample and reagents, simultaneously or sequentially. When cells (e.g., fixed cells or un-fixed cells) are loaded, the well may be subjected to washing, e.g., to remove excess cells from the well, microwell array, or plate. Similarly, washing may be performed to remove excess beads or other reagents from the well, microwell array, or plate. In addition, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

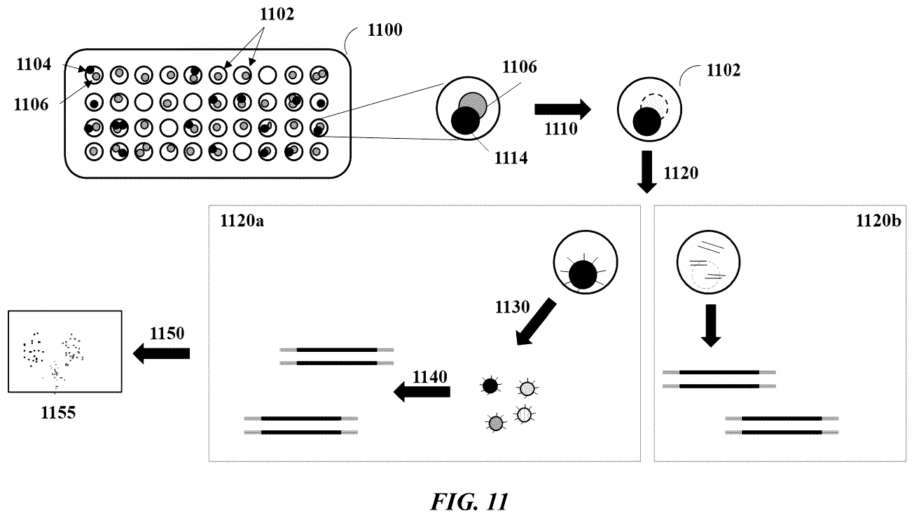

FIG. 11 schematically shows an example workflow for processing nucleic acid molecules within a sample. A substrate 1100 comprising a plurality of microwells 1102 may be provided. A sample 1106 which may comprise a cell (e.g., a fixed cell or an un-fixed cell), cellular components or analytes (e.g., proteins and/or nucleic acid molecules) can be co-partitioned, in a plurality of microwells 1102, with a plurality of beads 1104 comprising nucleic acid barcode molecules. During process 1110, the sample 1106 may be processed within the partition. For instance, the cell may be subjected to conditions sufficient to lyse the cells (e.g., fixed cells or un-fixed cells) and release the analytes contained therein. In process 1120, the bead 1104 may be further processed. By way of example, processes 1120a and 1120b schematically illustrate different workflows, depending on the properties of the bead 1104.

In 1120a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1130, the beads 1104 from multiple wells 1102 may be collected and pooled. Further processing may be performed in process 1140. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1150, further characterization, such as sequencing may be performed to generate sequencing reads. The sequencing reads may yield information on individual cells or populations of cells (e.g., fixed cells or un-fixed cells), which may be represented visually or graphically, e.g., in a plot 1155.

In 1120b, the bead comprises nucleic acid barcode molecules that are releasably attached thereto, as described below. The bead may degrade or otherwise release the nucleic acid barcode molecules into the well 1102; the nucleic acid barcode molecules may then be used to barcode nucleic acid molecules within the well 1102. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1150, further characterization, such as sequencing may be performed to generate sequencing reads. The sequencing reads may yield information on individual cells or populations of cells (e.g., fixed cells or un-fixed cells), which may be represented visually or graphically, e.g., in a plot 1155

In 1120b, the bead comprises nucleic acid barcode molecules that are releasably attached thereto, as described below. The bead may degrade or otherwise release the nucleic acid barcode molecules into the well 1102; the nucleic acid barcode molecules may then be used to barcode nucleic acid molecules within the well 1102. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1150, further characterization, such as sequencing may be performed to generate sequencing reads. The sequencing reads may yield information on individual cells or populations of cells (e.g., fixed cells or un-fixed cells), which may be represented visually or graphically, e.g., in a plot 1155.

### I. Additional Partition-Based Methods

The present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells or cell features may be used to characterize cells and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In other aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

For workflows comprising the use of fixation agents and/or un-fixing agents, labelling agents may be used to label samples (e.g., cells, fixed cells or un-fixed cells) at different points in time. In one embodiment, a plurality of cells is labeled prior to treatment with a fixation agent and/or after treatment with a fixation agent. In another embodiment, a plurality of fixed cells is labeled prior to treatment with an un-fixing agent and/or after treatment with an un-fixing agent. In one additional embodiment, a plurality of un-fixed cells is labeled prior to partitioning into partitions (e.g., wells or droplets) for further processing. In another embodiment, the methods, compositions, systems, and kits described herein provide labeled cells, labeled fixed cells or labeled un-fixed cells.

Labelling agents capable of binding to or otherwise coupling to one or more cells (including fixed cells and un-fixed cells) may be used to characterize a cell as belonging to a particular set of cells. For example, labeling agents may be used to label a sample of cells or a group of cells (including fixed cells and un-fixed cells). In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a cell (including a fixed or an un-fixed cell) may comprise subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 MM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 pM.

In another example, a reporter oligonucleotide may be coupled to a cell-penetrating peptide (CPP), and labeling cells may comprise delivering the CPP coupled reporter oligonucleotide into an analyte carrier. Labeling analyte carriers may comprise delivering the CPP conjugated oligonucleotide into a cell and/or cell bead by the cell-penetrating peptide. A CPP that can be used in the methods provided herein can comprise at least one non-functional cysteine residue, which may be either free or derivatized to form a disulfide link with an oligonucleotide that has been modified for such linkage. Non-limiting examples of CPPs that can be used in embodiments herein include penetratin, transportan, plsl, TAT (48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP may be an arginine-rich peptide transporter. The CPP may be Penetratin or the Tat peptide. In another example, a reporter oligonucleotide may be coupled to a fluorophore or dye, and labeling cells may comprise subjecting the fluorophore-linked barcode molecule to conditions suitable for binding the fluorophore to the surface of the cell. In some instances, fluorophores can interact strongly with lipid bilayers and labeling cells may comprise subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling cells may comprise delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane may be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus. In one embodiment, a reporter oligonucleotide coupled to a lipophilic molecule will remain associated with and/or inserted into lipid membrane (as described herein) via the lipophilic molecule until lysis of the cell occurs, e.g., inside a partition.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular cell population, or sample. Cell populations may be incubated with a plurality of libraries such that a cell or cells comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

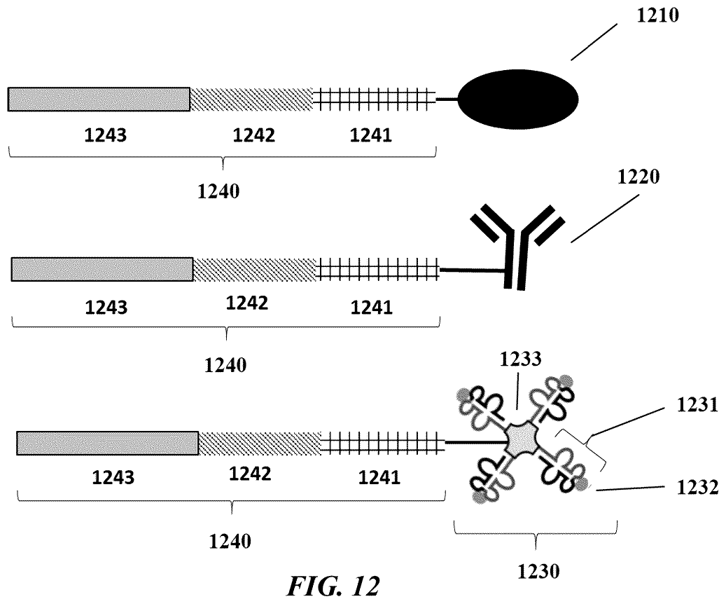

FIG. 12 describes exemplary labelling agents (1210, 1220, 1230) comprising reporter oligonucleotides (1240) attached thereto. Labelling agent 1210 (e.g., any of the labelling agents described herein) is attached (either directly, e.g., covalently attached, or indirectly) to reporter oligonucleotide 1240. Reporter oligonucleotide 1240 may comprise barcode sequence 1242 that identifies labelling agent 1210. Reporter oligonucleotide 1240 may also comprise one or more functional sequences 1243 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 12, in some instances, reporter oligonucleotide 1240 conjugated to a labelling agent (e.g., 1210, 1220, 1230) comprises a primer sequence 1241, a barcode sequence 1242 that identifies the labelling agent (e.g., 1210, 1220, 1230), and functional sequence 1243. Functional sequence 1243 may be configured to hybridize to a complementary sequence, such as a complementary sequence present on a nucleic acid barcode molecule 1290 (not shown), such as those described elsewhere herein. In some instances, nucleic acid barcode molecule 1290 is attached to a support (e.g., a bead, such as a gel bead), such as those described elsewhere herein. For example, nucleic acid barcode molecule 1290 may be attached to the support via a releasable linkage (e.g., comprising a labile bond), such as those described elsewhere herein. In some instances, reporter oligonucleotide 1240 comprises one or more additional functional sequences, such as those described above.

In some instances, the labelling agent 1210 is a protein or polypeptide (e.g., an antigen or prospective antigen) comprising reporter oligonucleotide 1240. Reporter oligonucleotide 1240 comprises barcode sequence 1242 that identifies polypeptide 1210 and can be used to infer the presence of an analyte, e.g., a binding partner of polypeptide 1210 (i.e., a molecule or compound to which polypeptide 1210 can bind). In some instances, the labelling agent 1210 is a lipophilic moiety (e.g., cholesterol) comprising reporter oligonucleotide 1240, where the lipophilic moiety is selected such that labelling agent 1210 integrates into a membrane of a cell or nucleus. Reporter oligonucleotide 1240 comprises barcode sequence 1242 that identifies lipophilic moiety 1210 which in some instances is used to tag cells (e.g., groups of cells, cell samples, etc.) and may be used for multiplex analyses as described elsewhere herein. In some instances, the labelling agent is an antibody 1220 (or an epitope binding fragment thereof) comprising reporter oligonucleotide 1240. Reporter oligonucleotide 1240 comprises barcode sequence 1242 that identifies antibody 1220 and can be used to infer the presence of, e.g., a target of antibody 1220 (i.e., a molecule or compound to which antibody 1220 binds). In other embodiments, labelling agent 1230 comprises an MHC molecule 1231 comprising peptide 1232 and reporter oligonucleotide 1240 that identifies peptide 1232. In some instances, the MHC molecule is coupled to a support 1233. In some instances, support 1233 may be a polypeptide, such as streptavidin, or a polysaccharide, such as dextran. In some instances, reporter oligonucleotide 1240 may be directly or indirectly coupled to MHC labelling agent 1230 in any suitable manner. For example, reporter oligonucleotide 1240 may be coupled to MHC molecule 1231, support 1233, or peptide 1232. In some embodiments, labelling agent 1230 comprises a plurality of MHC molecules, (e.g. is an MHC multimer, which may be coupled to a support (e.g., 1233)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. 10,550,429 and U.S. Pat. Pub. 20190367969, each of which is herein entirely incorporated by reference for all purposes.

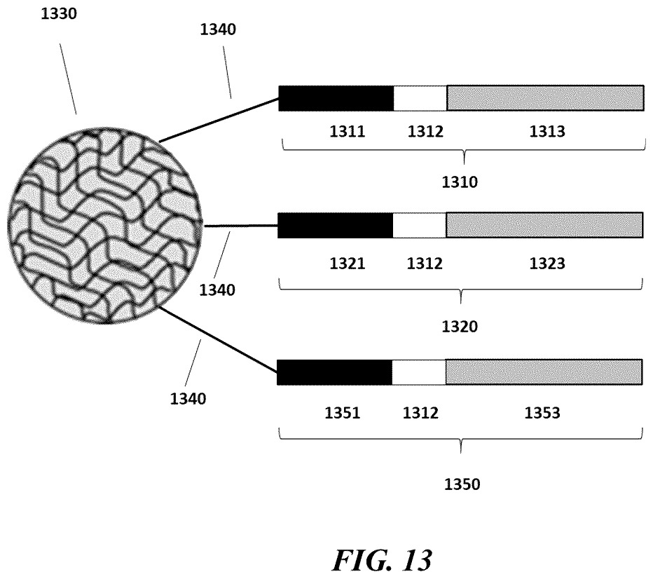

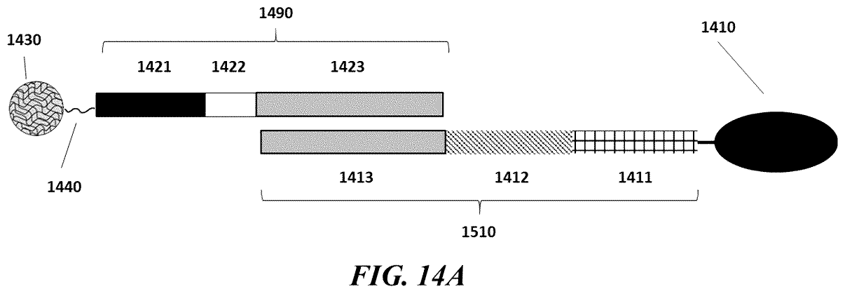

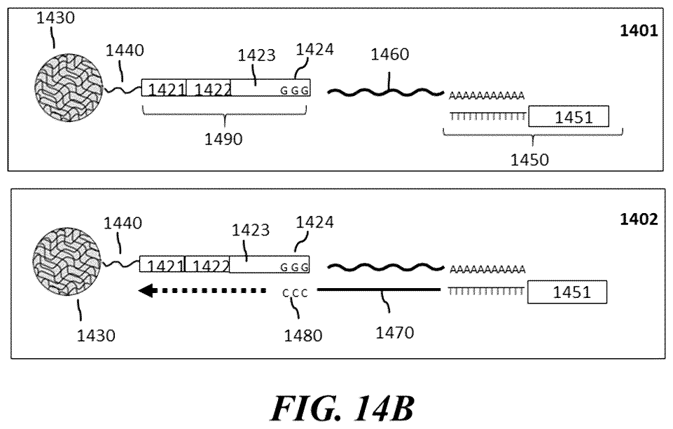

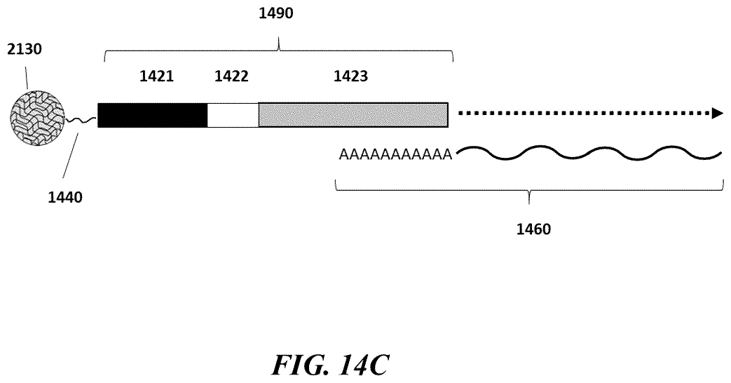

FIG. 13 illustrates another example of a barcode carrying bead. In some embodiments, analysis of multiple analytes (e.g., RNA and one or more analytes using labelling agents described herein) may comprise nucleic acid barcode molecules as generally depicted in FIG. 13. In some embodiments, nucleic acid barcode molecules 1310 and 1320 are attached to support 1330 via a releasable linkage 1340 (e.g., comprising a labile bond) as described elsewhere herein. Nucleic acid barcode molecule 1310 may comprise adapter sequence 1311, barcode sequence 1312 and adapter sequence 1313. Nucleic acid barcode molecule 1320 may comprise adapter sequence 1321, barcode sequence 1312, and adapter sequence 1323, wherein adapter sequence 1323 comprises a different sequence than adapter sequence 1313. In some instances, adapter 1311 and adapter 1321 comprise the same sequence. In some instances, adapter 1311 and adapter 1321 comprise different sequences. Although support 1330 is shown comprising nucleic acid barcode molecules 1310 and 1320, any suitable number of barcode molecules comprising common barcode sequence 1312 are contemplated herein. For example, in some embodiments, support 1330 further comprises nucleic acid barcode molecule 1350. Nucleic acid barcode molecule 1350 may comprise adapter sequence 1351, barcode sequence 1312 and adapter sequence 1353, wherein adapter sequence 1353 comprises a different sequence than adapter sequence 1313 and 1323. In some instances, nucleic acid barcode molecules (e.g., 1310, 1320, 1350) comprise one or more additional functional sequences, such as a UMI or other sequences described herein. The nucleic acid barcode molecules 1310, 1320 or 1350 may interact with analytes as described elsewhere herein, for example, as depicted in FIGS. 14A-C.

Referring to FIG. 14A, in an instance where cells are labelled with labeling agents, sequence 1423 may be complementary to an adapter sequence of a reporter oligonucleotide. Cells may be contacted with one or more reporter oligonucleotide 1510 conjugated labelling agents 1410 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 1410 which is conjugated to oligonucleotide 1510 and support 1430 (e.g., a bead, such as a gel bead) comprising nucleic acid barcode molecule 1490 is partitioned into a partition amongst a plurality of partitions (e.g., a droplet of a droplet emulsion or a well of a microwell array). In some instances, the partition comprises at most a single cell bound to labelling agent 1410. In some instances, reporter oligonucleotide 1510 conjugated to labelling agent 1410 (e.g., polypeptide, an antibody, pMHC molecule such as an MHC multimer, etc.) comprises a first adapter sequence 1411 (e.g., a primer sequence), a barcode sequence 1412 that identifies the labelling agent 1410 (e.g., the polypeptide, antibody, or peptide of a pMHC molecule or complex), and an adapter sequence 1413. Adapter sequence 1413 may be configured to hybridize to a complementary sequence, such as sequence 1423 present on a nucleic acid barcode molecule 1490. In some instances, oligonucleotide 1510 comprises one or more additional functional sequences, such as those described elsewhere herein.

Barcoded nucleic may be generated (e.g., via a nucleic acid reaction, such as nucleic acid extension or ligation) from the constructs described in FIGS. 14A-C. For example, sequence 1413 may then be hybridized to complementary sequence 1423 to generate (e.g., via a nucleic acid reaction, such as nucleic acid extension or ligation) a barcoded nucleic acid molecule comprising cell (e.g., partition specific) barcode sequence 1422 (or a reverse complement thereof) and reporter sequence 1412 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 14A-C, or a combination of workflows for an individual analyte, as described elsewhere herein. For example, by using a combination of the workflows as generally depicted in FIGS. 14A-C, multiple analytes can be analyzed.

In some instances, analysis of an analyte (e.g. a nucleic acid, a polypeptide, a carbohydrate, a lipid, etc.) comprises a workflow as generally depicted in FIG. 14A. A nucleic acid barcode molecule 1490 may be co-partitioned with the one or more analytes. In some instances, nucleic acid barcode molecule 1490 is attached to a support 1430 (e.g., a bead, such as a gel bead), such as those described elsewhere herein. For example, nucleic acid barcode molecule 1490 may be attached to support 1430 via a releasable linkage 1440 (e.g., comprising a labile bond), such as those described elsewhere herein. Nucleic acid barcode molecule 1490 may comprise a barcode sequence 1421 and optionally comprise other additional sequences, for example, a UMI sequence 1422 (or other functional sequences described elsewhere herein). The nucleic acid barcode molecule 1490 may comprise a sequence 1423 that may be complementary to another nucleic acid sequence, such that it may hybridize to a particular sequence.

For example, sequence 1423 may comprise a poly-T sequence and may be used to hybridize to mRNA. Referring to FIG. 14C, in some embodiments, nucleic acid barcode molecule 1490 comprises sequence 1423 complementary to a sequence of RNA molecule 1460 from a cell. In some instances, sequence 1423 comprises a sequence specific for an RNA molecule. Sequence 1423 may comprise a known or targeted sequence or a random sequence. In some instances, a nucleic acid extension reaction may be performed, thereby generating a barcoded nucleic acid product comprising sequence 1423, the barcode sequence 1421, UMI sequence 1422, any other functional sequence, and a sequence corresponding to the RNA molecule 1460.

In another example, sequence 1423 may be complementary to an overhang sequence or an adapter sequence that has been appended to an analyte. For example, referring to FIG. 14B, in some embodiments, primer 1450 comprises a sequence complementary to a sequence of nucleic acid molecule 1460 (such as an RNA encoding for a BCR sequence) from an analyte carrier. In some instances, primer 1450 comprises one or more sequences 1451 that are not complementary to RNA molecule 1460. Sequence 1451 may be a functional sequence as described elsewhere herein, for example, an adapter sequence, a sequencing primer sequence, or a sequence the facilitates coupling to a flow cell of a sequencer. In some instances, primer 1450 comprises a poly-T sequence. In some instances, primer 1450 comprises a sequence complementary to a target sequence in an RNA molecule. In some instances, primer 1450 comprises a sequence complementary to a region of an immune molecule, such as the constant region of a TCR or BCR sequence. Primer 1450 is hybridized to nucleic acid molecule 1460 and complementary molecule 1470 is generated. For example, complementary molecule 1470 may be cDNA generated in a reverse transcription reaction. In some instances, an additional sequence may be appended to complementary molecule 1470. For example, the reverse transcriptase enzyme may be selected such that several non-templated bases 1480 (e.g., a poly-C sequence) are appended to the cDNA. In another example, a terminal transferase may also be used to append the additional sequence. Nucleic acid barcode molecule 1490 comprises a sequence 1424 complementary to the non-templated bases, and the reverse transcriptase performs a template switching reaction onto nucleic acid barcode molecule 1490 to generate a barcoded nucleic acid molecule comprising cell (e.g., partition specific) barcode sequence 1422 (or a reverse complement thereof) and a sequence of complementary molecule 1470 (or a portion thereof). In some instances, sequence 1423 comprises a sequence complementary to a region of an immune molecule, such as the constant region of a TCR or BCR sequence. Sequence 1423 is hybridized to nucleic acid molecule 1460 and a complementary molecule 1470 is generated. For example, complementary molecule 1470 may be generated in a reverse transcription reaction generating a barcoded nucleic acid molecule comprising cell (e.g., partition specific) barcode sequence 1422 (or a reverse complement thereof) and a sequence of complementary molecule 1470 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

### EXAMPLES

Various features and embodiments of the disclosure are illustrated in the following representative examples, which are intended to be illustrative, and not limiting. Those skilled in the art will readily appreciate that the specific examples are only illustrative of embodiments of the disclosure as described more fully in the claims which follow thereafter. Every embodiment and feature described in the application should be understood to be interchangeable and combinable with every embodiment contained within.

### Example 1: Synthesis of a Reversible Fixing Reagent

This example illustrates the synthesis and method of use of a reversible fixing reagent of the present disclosure, ethane-1,2-diyl bis(1H-imidazole-1-carboxylate) (compound (2a)). The fixing reagent of compound (2a) reacts with the amine-bearing moieties of biomolecules in a biological sample to forms bis-carbamate crosslinks that are reversible upon treatment with an unfixing agent that cleaves carbamate bonds, such as DETA.

### Synthesis of Compound (2a): The Synthesis of Compound (2a) is Summarized in Scheme 7

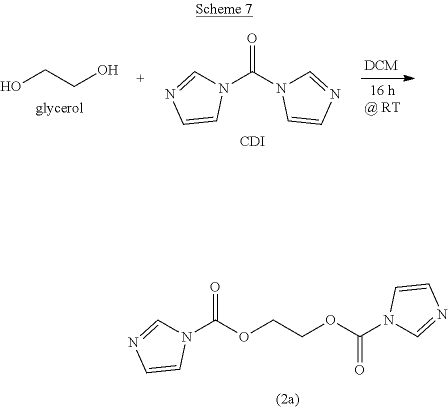

All reagents were obtained commercially (Millipore Sigma) and used without further purification. 20 g carbonyldiimdazole (“CDI”) (123 mmol, 3.0 eq.) was added to a 250 mL single neck RBF with stir bar. 100 mL dichloromethane (“DCM”) solvent was added, and the solution was rapidly stirred to afford a fine white suspension. Glycerol (2.30 mL, 40.1 mmol, 1 eq.) was added via syringe. The reaction vessel was sealed and topped with an Ar balloon to maintain an inert atmosphere. The reaction was allowed to proceed for 16 h and the solution turned from a white suspension to a clear pale yellow. The resulting solution was washed 3× with 100 mL deionized water and dried over MgSO₄. The solvent was removed under vacuum to provide a fine white/off-white powder. The formation of the desired product of compound (2a) was confirmed by NMR, which shows 3 peaks between 7.0-8.5 ppm and a single peak for the two CH₂ groups of the alkyl bridge. The resulting compound (2a) was used without further purification as a fixing reagent as described in Example 2.

Synthesis of Compound (5a): The Synthesis of bis-imidazole-glycerol (Compound (5a)) is Summarized in Scheme 8

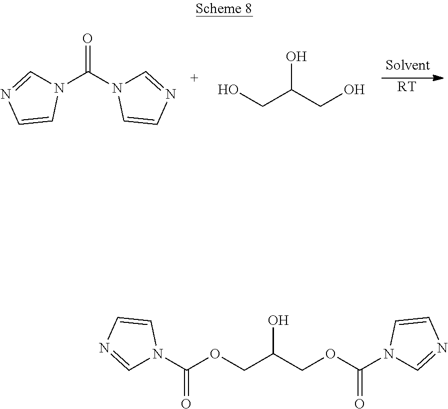

The bis-imidazole glycerol scaffold of compound (5a) is synthesized according to the conditions used for compound (2a) except that 2.5 equivalents of CDI in dichloromethane was added and the reaction allowed to proceed 16 h at RT. The formation of compound (5a) can be identified by ¹H NMR by integrating aryl peaks corresponding to imidazole (δ=7.00-8.5 ppm) and CH₂ peaks (δ=4.6 ppm) which integrate for 6H and 4H respectively.

### Synthesis of Compound (5b): The Synthesis of acetyl-bis-imidazole-glycerol (Compound (5b)) is Summarized in Scheme 9

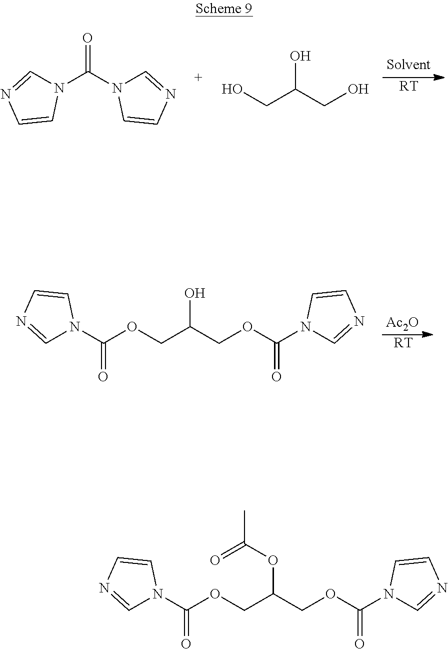

Compound (5b), the acetylated analogue of compound (5a), is synthesized according to the conditions described above for compound (2a) except that after 16 h, 1.5 eq. acetic anhydride is added, and the reaction is allowed to stir for an additional 4 h. The acetylated product is identified by the appearance of a CH₃ peak at δ=2.00 ppm and the broadening of multiplets corresponding to the glycerol backbone.

### Synthesis of Compound (5c): The Synthesis of tris-imidazole-glycerol (Compound (5c)) is Summarized in Scheme 10

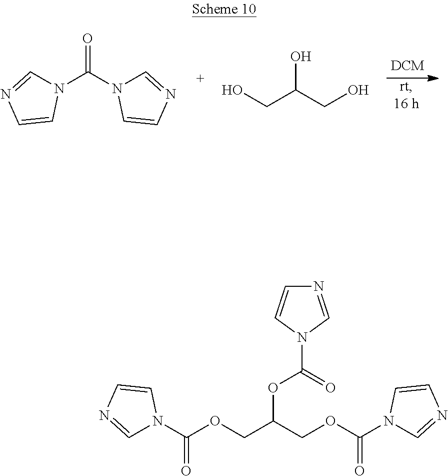 [SMILES: O=C(n1ccnc1)n1ccnc1 ; OCC(O)CO ; "rt,16 h" ; O=C(OCC(COC(=O)n1ccnc1)OC(=O)n1ccnc1)n1ccnc1]

Compound (5c) is synthesized according to the conditions described above for compound (2a) except with an increase to 4-fold excess CDI in dichloromethane. The reaction is allowed to proceed for 16 h at RT. The product is confirmed by the integration of 9 aromatic protons and 5 alkyl protons.

### Example 2: Use of a Reversible Fixing Reagent

This example illustrates the use of a reversible fixing reagent, compound (2a). The fixing reagent of compound (2a) reacts with the amine-bearing moieties of biomolecules in a biological sample to forms bis-carbamate crosslinks that are reversible upon treatment with an unfixing agent that cleaves carbamate bonds, such as DETA. This example illustrates the use of compound (2a) to fix PBMCs, which were then stored for up to 21 days followed by un-fixing with DETA to yield RNA.

Fixation of PBMCs: Compound (2a), prepared as described in Example 1, was formulated as a 200 mM stock solution in anhydrous DMSO. The final target fixative solutions were prepared by dilution of the stock solution in PBS to provide the desired concentration. The formulated fixative solutions of compound (2a) were used within 10 mins of formulation to prevent hydrolysis of the reactive imidazole-carboxylates. PBMCs were fixed in 100 μL of 15 mM fixative solution of compound (2a) for either 30 min or 1 h, then quenched with 10% Fetal Bovine Serum (FBS) in PBS. For comparison, separate PBMC samples were fixed in 1 mL of 4% paraformaldehyde (“PFA”) in PBS for 20 min and quenched with 10% FBS in PBS. The fixed PBMCs were stored for up to 21 days at either RT, 4° C., or −80° C.

RNA assay of un-fixed PBMCs: At weekly time points, cells from the fixed PBMC samples were centrifuged at 450 g for 5 min, and supernatant was collected and retained. The pellet of the compound (2a)-fixed PBMCs was un-fixed (or “de-crosslinked”) by incubation in solutions of 0.1% diethylenetriamine (DETA) in PBS at 50° C. for 15 min or with 0.1% SDS in PBS/0.3% fos-choline in PBS at 40° C. for 2 h. The pellet of the PFA-fixed PBMCs was un-fixed by incubation in 0.1% SDS, 30 mM Tris, pH 6.8 at 40° C. for 2 h. After unfixing agent treatment, the PBMCs were centrifuged at 450 g for 5 min and both pellet (if any) and supernatant were collected.

RNA was isolated from the supernatant obtained before treatment with unfixing agent (labeled “storage solution”) and the supernatant collected after the unfixing agent treatment (labeled “supernatant”) was isolated using the RNAeasy MinElute Cleanup kit (Qiagen, Cat #74204). RNA from the cell pellets obtained after unfixing agent treatment (“pellet”) was isolated using the RNAeasy Plus Mini Kit (Qiagen, Cat #74134). The isolated RNA was evaluated by Qubit™ RNA HS Assay Kit (Invitrogen, Cat #Q32855) and Agilent RNA Screen Tape System (Agilent Technologies).

Results: As shown by the results in Table 3, all samples, fresh or treated with fixing reagent, showed significant leakage of RNA into the storage solution during storage. The samples treated with fixing reagent for 60 minutes and stored for 21 days showed significantly higher RNA recovery from the Pellet and Supernatant with relatively low leakage even after 21 days storage.

TABLE 3

| | Conditions | | | | | | | | | | |
|---|---|---|---|---|---|---|---|---|---|---|---|
| | Fixation | Storage | Pellet | | | Supernatant | | | Storage Solution | | |
| | time | time | RNA | | | RNA | | | RNA | | |
| Sample | (min) | (day) | (ng) | SD | DV200 | (ng) | SD | DV200 | (ng) | SD | DV200 |
| Fresh | 0 | 0 | 159.3 | 0 | 80.1 | | | | 203 | 0 | 66.6 |
| Fresh, 4 C | 0 | 7 | 340 | 108 | 84.7 | | | | 165 | 68.3 | 71.9 |
| Fresh, 4 C | 0 | 14 | 9.4 | 2.4 | 67.4 | | | | 63.1 | 13.6 | 54.6 |
| 15 mM, 4 C | 30 | 0 | 21.4 | 11.8 | 87.6 | 526 | 140 | 93.8 | | | |
| 15 mM, 4 C | 30 | 7 | 18.4 | 3.2 | 91.5 | 54.0 | 46.1 | 91 | 130 | 95.4 | 87.5 |
| 15 mM, 4 C | 30 | 14 | 17.2 | 0.84 | 48.8 | 85.0 | 7.4 | 61.6 | 121 | 20.3 | 68.2 |
| 15 mM, 4 C | 30 | 21 | 16.2 | 5.6 | 81.3 | 363 | 21.8 | 80.7 | 260 | 54.4 | 71.3 |
| 15 mM, 4 C | 30 | 21 | 16.2 | 5.6 | 81.3 | 363 | 21.8 | 80.7 | 260 | 54.4 | 71.3 |
| 15 mM, 4 C | 60 | 21 | 211 | 55.8 | 76.0 | 307 | 1.98 | 79.4 | 98.6 | 26.0 | 56.5 |

### Example 3: Solvent Conditions for Use of a Reversible Fixation Reagent

The results of Example 2 suggest some leakage of RNA from PBMCs fixed with compound (2a) and it was hypothesized that the solvent conditions used for fixation, particularly the amount of DMSO present, may contribute to this leakage. This example illustrates experimental studies to determine optimal solvent conditions for use of the reversible fixation reagent, compound (2a), in fixing a biological sample of PBMCs.

Materials and Methods: Compound (2a), was prepared as described in Example 1, and formulated as a 200 mM stock solution in anhydrous DMSO. Fixative solutions of compound (2a) were prepared by dilution of the stock solution in PBS at the following fixative reagent and DMSO concentrations: 15 mM compound (2a) in 15% DMSO; 15 mM compound (2a) in 10% DMSO; 15 mM compound (2a) in 7.5% DMSO; 10 mM compound (2a) in 5% DMSO; or 5 mM compound (2a) in 2.5% DMSO. Fresh PBMCs were fixed in 100 μL of each of the range of fixative solutions for either 30 min or 1 h, then quenched with 100 mM Tris followed by a wash in 10% FBS in PBS.

At Day 0 and Day 3 time points, cells from the fixed PBMC samples were centrifuged at 450 g for 5 min, and supernatant was collected and retained. The pellet of the compound (2a)-fixed cells was un-fixed by incubation in solutions of 1% diethylenetriamine (DETA). After unfixing agent treatment, the samples were centrifuged at 450 g for 5 min and both pellet (if any) and supernatant were collected. RNA was isolated from the supernatant obtained before treatment with unfixing agent (labeled “storage solution”) and the supernatant collected after the unfixing agent treatment (labeled “supernatant”) was isolated using the RNAeasy MinElute Cleanup Kit (Qiagen, Cat #74204). RNA from the cell pellets obtained after unfixing agent treatment (“pellet”) was isolated using the RNAeasy Plus Mini Kit (Qiagen, Cat #74134). RNA isolated was evaluated for mass recovery and quality (Qubit or Tapestation respectively).

Results: It was observed that lower DMSO concentration during fixation with compound (2a) resulted in improved amounts of RNA retention. For example, fixation of cells in the sample with a 2.5% DMSO solution of only 5 mM compound (2a) resulted in significantly higher RNA retention after 3 days at 4C compared to fixation with a 15 mM solution of compound (2a) in 10% or 15% DMSO.

### Example 4: Preparation of a Reversibly Fixed Biological Sample in a Discrete Droplet With a Carbamate-Reversing Unfixing Agent

This example illustrates preparation of discrete droplets (GEMs) containing a biological sample of PBMCs previously fixed with compound (2a), and the carbamate-cleaving unfixing agent DETA, and then performing a single-cell RNA sequence expression profiling experiment using the un-fixed samples in the droplets.

### Preparation of Fixed Biological Sample:

A fixed biological sample of fixed PBMCs is prepared as described above in Example 1. The fixed biological sample can be stored at 4C or −20 C for several days or more before being processed in a droplet-based assay (e.g., a single cell assay).

### Preparation of Unfixing Agent:

A 0.1% stock solution of the unfixing agent DETA is prepared as described in Example 2.

### Generation of Droplets (GEMs) With Fixed Cells, Unfixing Agent, and Barcoded Gel-Beads

The fixed biological sample comprising fixed PBMCs is changed into the standard master mix used with the Chromium System (10× Genomics, Pleasanton, CA, USA) for partitioning samples together with barcoded gel beads in discrete droplets called GEMs (“Gel Beads in Emulsion”). The Chromium System is prepared with the unfixing agent solution added as a separate reagent in generating the GEM containing the sample PBMC and the barcode gel bead. Alternatively, the unfixing agent solution is added to the reservoir containing the suspension of barcoded gel-beads and introduced into the GEMs through the same inlet channel with the gel-beads. Once generated, the GEMs are collected, and a heat incubation step is carried out. The heating step facilitates lysis and release of the cell contents, barcode oligonucleotides, and the RT reaction that results in the cDNA synthesis reaction incorporating the barcodes in the 3′ synthons. In incorporating an unfixing agent with the GEMs, the heat incubation step can be extended as necessary to allow for the un-fixing reaction of the DETA that removes the carbamate crosslinks from biomolecules released from the PBMC sample in the GEM.

### Example 5: Reversible Fixation With Compound (2a) and the Enzymatic Unfixing Agent, Carboxyesterase (CES)

This example illustrates the use of compound (2a) to fix Jurkats cells, which were then stored for up to 3 days followed by un-fixing using carboxyesterase to yield RNA.

### Materials and Methods:

### A. Preparation of Stock Solutions:

The fixative of compound (2a), was prepared as described in Example 1. A 200 mM stock solution of compound (2a) was prepared in anhydrous DMSO.

Stock solutions of the small molecule unfixing agents, EDA, hydrazine, FosCholine, glycine, and ethanolamine were prepared as follows. EDA, hydrazine, or ethanolamine was diluted in 50 mM Tris buffer, pH 8,3 to make 0.1% w/v solution. Glycine was diluted in 50 mM Tris buffer, pH 8,3 to make 100 mM solution. In some groups, FosCholine was added to provide a final concentration of 2%. In each prepared solution, Qiagen RNAse inhibitor (Qiagen, Cat #129916) was included with a final concentration of 1 unit/μL.

A stock solution of the enzymatic unfixing agent, carboxyesterase (“CES”) was prepared as follows. Carboxylesterase 1 or 2 was diluted in 50 mM Tris buffer, pH 8.3 to make final concentration 1 unit/μL. In some groups, FosCholine was added to provide a final concentration of 2%. In each prepared solution, Qiagen RNAse inhibitor (Qiagen, Cat #129916) was included with a final concentration of 1 unit/μL.

### B. Fixation of Jurkats:

The final target fixative solutions were prepared by dilution of the stock solution in PBS to provide a concentration of 10 mM. The formulated fixative solutions of compound (2a) were used within 10 mins of formulation to prevent hydrolysis of the reactive imidazole-carboxylates. Fresh Jurkats cells were fixed in 100 μL of the 10 mM fixative solution of compound (2a) for 1h, then quenched with 10% Fetal Bovine Serum (FBS) in PBS.

### C. Unfixing Agent Treatment of Fixed Cells:

At Day 0 and Day 3 time points, cells from the fixed samples were centrifuged at 300 g for 5 min, and supernatant was collected and retained. The pellet of the compound (2a)-fixed cells was un-fixed by incubation with carboxyesterase or the small-molecule unfixing agents as follows. Cell pellets were resuspended and incubated with un-fixing solution containing EDA, hydrazine, ethanolamine, or hydrazine with and without FosCholine at 50° C. for 15 min. Cell pellets were resuspended and incubated with un-fixing solution containing CES 1 or 2 with and without FosCholine at 37° C. for 30 min.

### D. Extracted RNA Quantitation

After unfixing agent treatment, the samples were centrifuged at 300 g for 5 min and both pellet (if any) and supernatant were collected. RNA was isolated from the supernatant obtained before treatment with unfixing agent (labeled “storage solution”) and the supernatant collected after the unfixing agent treatment (labeled “supernatant”) was isolated using the RNAeasy MinElute Cleanup Kit (Qiagen, Cat #74204). RNA from the cell pellets obtained after unfixing agent treatment (“pellet”) was isolated using the RNAeasy Plus Mini Kit (Qiagen, Cat #74134). RNA isolated was evaluated for mass recovery and quality (Qubit or Tapestation respectively).

Results: As shown in Table 4, use of the enzymatic unfixing agent, carboxyesterase resulted in much higher RNA recovery from cells fixed with compound (2a) (relative to Fresh cells) than other small molecule unfixing agents, including EDA, hydrazine, FosCholine, glycine, and ethanolamine. Indeed, the total RNA recovered was comparable to the amount recovered from fresh cells that were not fixed.

TABLE 4

| | | Total Recovered RNA |
|---|---|---|
| Sample | Unfixing Agent Treatment | (ng) |
| Fresh | | 1237.5 |
| Fixed | None | 84.2 |
| Fixed | 0.1% EDA | 147.2 |
| Fixed | 0.1% EDA + 2% FosCholine | 97.7 |
| Fixed | 0.1% hydrazine | 242.6 |
| Fixed | 0.1% hydrazine + 2% FosCholine | 223.1 |
| Fixed | 100 mM glycine | 136.4 |
| Fixed | 100 mM glycine + 2% FosCholine | 391.5 |
| Fixed | 0.1% ethanolamine | 232.2 |
| Fixed | 0.1% ethanolamine + 2% FosCholine | 75.9 |
| Fixed | 1 unit/μL CES1 | 857.3 |
| Fixed | 1 unit/μl CES 1 + 2% FosCholine | 1458.0 |
| Fixed | 1 unit/μL CES 2 | 316.34 |
| Fixed | 1 unit/μl CES 2 + 2% FosCholine | 0 |

### Example 6: Recovery of High Quality RNA From Cells Fixed With Compound (2a) Without Use of an Unfixing Agent

This example illustrates the use of compound (2a) to fix Jurkats cells, which are then stored for up to 3 days in a storage buffer solution, lysed to extract RNA, without the use of an unfixing agent, and 3′-sequenced to determine quality of the extracted RNA.

### Materials and Methods

### A. Preparation of Stock Solutions:

A stock solution of the fixative of compound (2a), was prepared as described in Example 5.

Storage buffers solutions of sucrose, SSC, and BSA in PBS solutions were prepared at the following concentrations. The CellCover storage buffer solution was obtained from Anacyte Laboratories. The 50% sucrose storage buffer solution was prepared by dissolving sucrose in PBS to provide a final concentration of 50% w/v. The 20×SSC storage buffer solution was purchased from Sigma Aldrich (Sigma Cat #S6639) and diluted 3× in PBS before use. The 0.04% BSA in PBS storage buffer solution was prepared by diluting 10% BSA (Miltenyi Biotec Cat #130-091-376) in PBS to a final concentration of 0.04%.

### B. Fixation of Jurkats:

Fixative solutions were prepared by dilution of the compound (2a) stock solution in PBS to provide a concentration of 10 mM compound (2a). The formulated fixative solutions of compound (2a) were used within 10 mins of formulation to prevent hydrolysis of the reactive imidazole-carboxylates. Fresh Jurkats cells were fixed in 100 μL of the 10 mM fixative solution of compound (2a) for 1 h, then quenched with 10% Fetal Bovine Serum (FBS) in PBS. The fixed cells were centrifuged at 300 g for 5 min to remove supernatant and then resuspended in 100 μL of storage buffer solution selected from CellCover, 50% sucrose, 3×SSC, or 0.04% BSA in PBS, and stored at 4° C. for 3 days.

### C. Single Cell 3′ Gene Expression Analysis

The fixed cells in storage buffer solutions were centrifuged at 300 g for 5 min to remove supernatant then resuspended in 100 μL PBS. The resuspended cells were then processed and sequenced according to the 10× Genomics Single Cell Gene Expression protocol (10× Genomics, Pleasanton, CA, USA).

Results: As shown by the results in Table 5, Jurkats cells fixed with compound (2a), stored for 3 days at 4° C. in 3×SSC or 50% sucrose storage buffer solution, followed by gene expression sample preparation using the 10× Genomics Single Cell Gene Expression protocol, provide high quality gene expression data comparable to data obtained from fresh cells. Significantly, the Pearson's Correlation (R2) between the Day 0 fresh cells and the Day 3 compound (2a)-fixed cells in SSC or sucrose storage buffer was 0.918 and 0.897, respectively. It is also surprising that the expression protocol did not include unfixing agent, and only a lysis agent. The ability to obtain high quality gene expression data from the fixed cells without the use of an unfixing agent indicates that treatment of cells with the fixing reagent of compound (2a) results in preferential fixation of cellular proteins that cause cellular degradation with a substantial amount of mRNA remaining unfixed yet preserved.

TABLE 5

| | | | | Pearson's |
|---|---|---|---|---|
| | | | | Correlation |
| | | | GRCh38 | (R²) |
| | Fraction | Fraction | Median | of Gene |
| | of Reads | of Reads | Genes | Expression |
| Sample | Total | Usable | per Cell | w/Fresh Cells |
| Fresh Cells | | | | |
| Day 0 | 68.3% | 41.7% | 2444 | N/A |
| Day 3 + CellCover | 80.5% | 30.6% | 591 | 0.733 |
| Day 3 + PBS | 29.9% | 14.4% | 907 | 0.750 |
| Day 3 + sucrose | 18.4% | 8.5% | 693 | 0.781 |
| Fixed Cells | | | | |
| Day 0 | 81.3% | 42.6% | 1937 | 0.819 |
| Day 3 + CellCover | 72.1% | 9.7% | 1054 | 0.765 |
| Day 3 + PBS | 57.3% | 17.3% | 1108 | 0.834 |
| Day 3 + SSC | 67.3% | 31.4% | 1877 | 0.918 |
| Day 3 + sucrose | 70.2% | 26.6% | 1797 | 0.897 |

### Example 7: Recovery of High Quality RNA From Cells Fixed With Compound (2a) and Un-fixed With CES and Hydrazine

This example illustrates the use of compound (2a) to fix PBMCs, which are then stored for up to 3 days in a storage buffer solution, un-fixed with a mixture of CES and hydrazine, and then 3′-sequenced to determine quality of the extracted RNA.

### Materials and Methods

### A. Preparation of Stock Solutions:

The fixative of compound (2a), was prepared as described in Example 1. A 200 mM stock solution of compound (2a) was prepared in anhydrous DMSO.

A stock solution of the enzymatic unfixing agent, carboxyesterase (“CES”) was obtained from Creative Biomart (Cat #CES1-29351TH) with a concentration of 20 ng/μL.

A stock solution of the small molecule unfixing agent, hydrazine was prepared by diluting hydrazing in 50 mM Tris buffer, pH 8.3 to make 10% w/v solution.

### B. Storage Buffer Solution

A storage buffer solution of 0.04% BSA and 5% Superase in PBS was prepared by dissolving RNase free BSA (Cat #B6917; Sigma-Aldrich) in PBS to a final concentration of 0.04%. Superase (Cat #AM2694; Thermo Fisher Scientific) then was added to a concentration of 5%.

### C. Fixation of PBMCs

Fixative solutions were prepared by dilution of the compound (2a) stock solution in PBS to provide a concentration of 10 mM or 20 mM compound (2a). The formulated fixative solutions of compound (2a) were used within 10 mins of formulation to prevent hydrolysis of the reactive imidazole-carboxylates. Fresh PBMCs were fixed in 100 μL of the 10 mM or 20 mM fixative solution of compound (2a) for 1 h, then quenched with 3% RNase BSA in PBS. The fixed cells were centrifuged at 300 g for 5 min to remove supernatant and then resuspended in 100 μL of storage buffer solution of 0.04% BSA in PBS with 5% Superase at 4° C. for 3 days.

### D. Partitioning and Single Cell 3′ Gene Expression Analysis

At Day 0 and Day 3 time points, cells from Fresh and Fixed samples were collected, counted, and suspended into the Single Cell 3′V3 protocol standard master mix used with the Chromium System (10× Genomics, Pleasanton, CA, USA). CES was added to the same master mix to make the final concentration 0.01 ng/μL. Meanwhile, hydrazine was added to barcoded gel beads (10× Genomics, Pleasanton, CA, USA) to make the final w/v concentration 0.1%. Both master mix and gel beads were loaded into the Chromium System (10× Genomics, Pleasanton, CA, USA) for partitioning samples together with barcoded gel beads in discrete droplets called GEMs (“Gel Beads in Emulsion”). Once generated, the GEMs are collected, and a heat incubation step is carried out. The heating step facilitates release of the cell contents and RNA, capture of RNA by barcode oligonucleotides, and the reverse-transcription (RT) reaction that results in cDNA synthesis incorporating the barcodes in the 3′ synthons.

Determination and mapping of PBMC cell types present in the samples was performed by automated meta-analysis of cell clusters identified using differentially expressed marker gene expression. PBMC cell type composition was identified by an automated script that quantifies the number and fraction of cell types known to be detected in PBMC samples by categorizing cells based on a combination of differentially expressed known marker genes for each cell type, with unclassified cells going to the undetermined category.

Results: As shown by the results in Table 6, PBMCs fixed with 10 mM or 20 mM compound (2a), stored for up to 3 days at 4° C., un-fixed using CES and hydrazine, and then prepared and analyzed using the 10× Genomics Single Cell Gene Expression protocol, provide high quality gene expression data comparable to data obtained from fresh cells. As shown in Table 6, the fixation preserves ˜60% UMI counts per cell and more than 90% median gene per cell after 3 days. Additionally, the Pearson's Correlation (R2) between the Day 0 fresh cells and the Day 3 compound (2a)-fixed cells, respectively.

TABLE 6

| | | | | Pearson's |
|---|---|---|---|---|
| | | | | Correlation |
| | Relative | Relative | GRCh38 | (R²) |
| | Median | Median | Median | of Gene |
| | UMI | Genes | Genes | Expression |
| Sample | per Cell | per Cell | per Cell | w/Fresh Cells |
| Fresh Cells | | | | |
| Day 0 | n/a | n/a | 1800 | n/a |
| Day 3 | 32% | 47% | 849 | 0.85 |
| 10 mM (2a) | | | | |
| Fixed Cells | | | | |
| Day 0 | 77% | 100% | 1798 | 0.92 |
| Day 3 | 51% | 83% | 1499 | 0.94 |
| 20 mM (2a) | | | | |
| Fixed Cells | | | | |
| Day 0 | 53% | 91% | 1635 | 0.90 |
| Day 3 | 56% | 91% | 1641 | 0.88 |

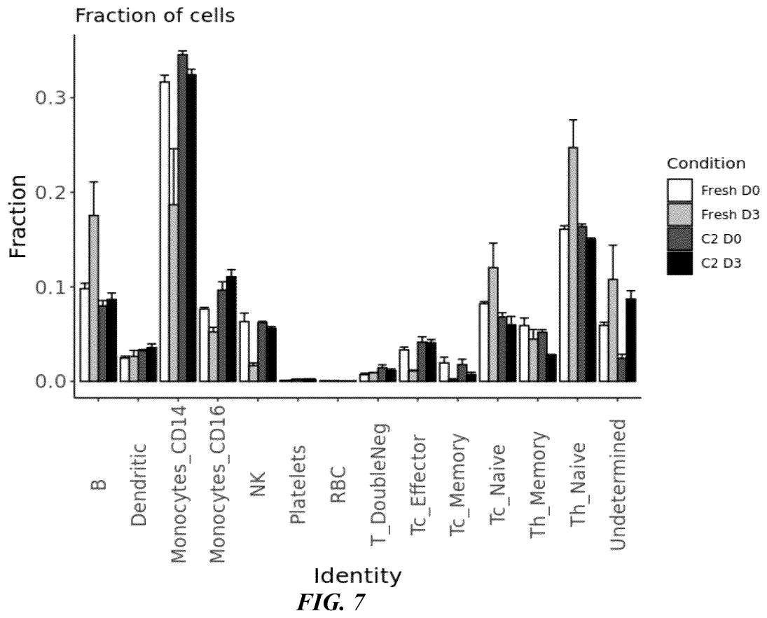

Additionally, as shown by the plot depicted in FIG. 7, cell counting was carried out to determine the proportion of different PBMC cell types found in the Fresh PBMCs as compared to the PBMCs fixed with compound (2a), stored, and then subjected to the un-fixing treatment of CES and hydrazine. It was observed that the proportions of B cells, T cells, monocyte cells, and undetermined cell populations found in the Fresh cell sample was similar to the proportions found in the fixed cell samples stored for 3 days then subjected to the un-fixing treatment. These comparative PBMC cell counting results indicate that fixation storage and then unfixing of cells using compounds and methods of the present disclosure can be used to analyze relatively cell populations from fixed and stored samples in a droplet-based assay.

The ability to obtain high quality gene expression data from the PBMCs fixed with compound (2a) and un-fixed using CES and hydrazine indicates RNA information in PBMCs can be preserved and retrieved with high efficiency using compound (2a) and the un-fixing conditions described above.

While the foregoing disclosure of the present disclosure has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the disclosure are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A method for preparing a biological sample comprising contacting the sample with a first fixing reagent composition comprising a compound of formula (I) 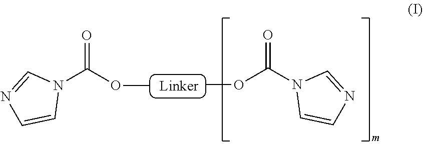
wherein, “Linker” comprises a linear or branched alkane moiety of 2-24 carbons; and m is 1 to 12.
